(19) United States
(12) Patent Application Publication
(10) Pub. No.: US 2026/0165708 A1
(43) Pub. Date: Jun. 18, 2026

(54) DRUG ELUTING NERVE GUIDE CONDUIT

(71) Applicant: New York University in Abu Dhabi Corporation, Abu Dhabi (AE)

(72) Inventors: Jiarui Zhou, Abu Dhabi (AE); Vijayavenkataraman Sanjairaj, Abu Dhabi (AE)

(21) Appl. No.: 18/707,382

(22) PCT Filed: Nov. 3, 2022
(86) PCT No.: PCT/IB2022/000642

§ 371 Date: May 3, 2024

Related U.S. Application Data
(60) Provisional application No. 63/275,197, filed on Nov. 3, 2021.

(51) Int. Cl. A61B 17/11; A61B 17/00; A61L 29/14; A61L 29/16

(52) U.S. Cl. CPC A61B 17/1128; A61L 29/145; A61L 29/16; A61B 2017/00893

(57) ABSTRACT

The present invention provides drug release system embedded nerve guidance conduits (NGC) devices. The devices improve nerve regeneration performance by providing long-term release of growth factors. The devices comprise intermediate cavities introduced along a length of a multi-channel NGC that act as relays to enhance regenerative cell concentrations or growth factor delivery to improve regeneration performance. In some embodiments, the devices target peripheral nerve injury treatments.

### CROSS-REFERENCE TO RELATED APPLICATIONS

This application claims priority to U.S. Provisional Application No. 63/275,197, filed on Nov. 3, 2021, incorporated herein by reference in its entirety.

### BACKGROUND OF THE INVENTION

Nerve guidance conduits (NGCs) are tubular tissue engineering scaffolds that act as a bridge between the proximal and distal ends of native nerve to facilitate nerve regeneration. The application of NGCs is mostly limited to nerve defects less than 3 mm in size due to the lack of sufficient cells in the lumen. Nerve regeneration over long gaps can be induced or enhanced using two main methods: introduction of nerve cells or nerve-related cells, like Schwann cells; and incorporating nerve growth factors. However, the traditional multichannel design of NGCs limits the use of both methods due to dimensional constraints as NGC diameters typically start from 2 mm, with each of the multichannel diameters ranging within a few hundred microns.

Thus, there is a need in the art for improved nerve guidance conduit devices for enhanced nerve regeneration and repair. The present invention satisfies this need.

### SUMMARY OF THE INVENTION

In some aspects, a nerve guidance conduit device, having: a tubular outer housing having an inner lumen; and a plurality of nerve-holding sections positioned within the inner lumen of the housing, each nerve-holding section comprising one or more channels each configured to receive a nerve; wherein the inner lumen of the housing is divided into an alternating series of nerve-holding sections and inner lumen cavities.

In some embodiments, the outer housing and the plurality of nerve-holding sections comprises a plurality of pores, such that each channel and each inner lumen cavity is fluidly connected to each other and to an exterior of the outer housing. In some embodiments, the device has at least one therapeutic-releasing substrate positioned within the inner lumen. In some embodiments, the substrate is chosen from a group consisting of: microparticles, bulk hydrogel and hydrogel film.

In some embodiments, the substrate is loaded with a therapeutic selected from the group consisting of: growth factors, neurotrophic factors, cell adhesion molecules, proteins, peptides, small molecules, nucleic acid molecules, cytokines, stem cells, Schwann cells, and upregulators of regeneration-associated genes. In some embodiments, the growth factor is glial cell line-derived neurotrophic factor (GDNF). In some embodiments, the substrate is configured to release the therapeutic over a period of time. In some embodiments, the substrate is configured to release the therapeutic over a period of at least 50 days.

In some embodiments, the one or more channels have diameters ranging from 50 μm to 1000 μm. In some embodiments, the outer housing has a diameter of at least 0.1 mm. In some embodiments, the outer housing has a length of a least 3 mm. In some embodiments, the hydrogel film has a thickness between 0.1 mm and 0.2 mm. In some embodiments, the microparticles comprise microspheres with diameters of about 100 μm. In some embodiments, the bulk hydrogel has a thickness of at least 25 μm. In some embodiments, the bulk hydrogel has a diameter of at least 100 μm. In some embodiments, the at least one therapeutic-releasing substrate is positioned in the inner lumen in a location chosen from a front, middle and/or back locations.

In some embodiments, the device comprises a material selected from the group consisting of: poly(lactic co-glycolic acid) (PGA), poly(1-lactic-acid) (PLA), polycaprolactone (PCL), poly(ethylene glycol) diacrylate (PEGDA), gelatin methacryloyl (GelMA), polydimethylsiloxane (PDMS), collagen, chitosan, decellularized extracellular matrix (dECM), cellulose, silk, and ionic liquids. In some embodiments, the device further comprises a coating on at least a portion of the device. In some embodiments, the coating is a material selected from the group consisting of: conductive materials, ferromagnetic materials, thermoelectric materials, nanoparticles, 2D materials such as graphene, MXenes, conductive polymers and hydrogels.

In some aspects, a method of treating nerve injury comprising implanting the device of claim 1 at the site of a nerve injury; thereby treating the injury.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A through FIG. 1D depict different views of an exemplary multichannel NGC. (FIG. 1A) standard view. (FIG. 1B) side view. (FIG. 1C) cross section view. (FIG. 1D) cross section perspective view.

FIG. 2A through FIG. 2D depict different types of drug release systems. (FIG. 2A) single layer microsphere system. (FIG. 2B) double layer microsphere system. (FIG. 2C) bulk hydrogel system. (FIG. 2D) hydrogel film system. (Grey: fluid region; Red: drug release system; White: wall region).

FIG. 3A through FIG. 3F depicts GDNF volume fraction of different drug release systems under the assumption of constant simulation time (519 s). (FIG. 3A) comparison among single, double layer microsphere and bulk hydrogel systems. (FIG. 3B-FIG. 3E) comparison within single layer microsphere only. (FIG. 3F) comparison between three drug release systems and the combined model.

FIG. 4A through FIG. 4P depict GDNF distribution of different drug release systems under the assumption of constant simulation time. (FIG. 4A) single layer microsphere system (microsphere features: quantity: 8; diameter: 0.1 mm; adjacent distance: 0.2 mm; position: middle). (FIG. 4B) single layer microsphere system (microsphere features: quantity: 8; diameter: 0.1 mm; adjacent distance: 0.2 mm; position: front). (FIG. 4C) single layer microsphere system (microsphere features: quantity: 8; diameter: 0.1 mm; adjacent distance: 0.2 mm; position: back). (FIG. 4D) single layer microsphere system (microsphere features: quantity: 24; diameter: 0.1 mm; adjacent distance: 0.2 mm; position: front+middle+back). (FIG. 4E) single layer microsphere system (microsphere features: quantity: 16; diameter: 0.1 mm; adjacent distance: 0.2 mm; position: front+back). (FIG. 4F) single layer microsphere system (microsphere features: quantity: 16; diameter: 0.1 mm; adjacent distance: 0.2 mm; position: front+middle). (FIG. 4G) single layer microsphere system (microsphere features: quantity: 16; diameter: 0.1 mm; adjacent distance: 0.2 mm; position: middle+back). (FIG. 4H) single layer microsphere system (microsphere features: quantity: 6; diameter: 0.1 mm; adjacent distance: 0.2 mm; position: middle). (FIG. 4I) single layer microsphere system (microsphere features: quantity: 4; diameter: 0.1 mm; adjacent distance: 0.2 mm; position: middle). (FIG. 4J) single layer microsphere system (microsphere features: quantity: 8; diameter: 0.15 mm; adjacent distance: 0.2 mm; position: middle). (FIG. 4K) single layer microsphere system (microsphere features: quantity: 8; diameter: 0.125 mm; adjacent distance: 0.2 mm; position: middle). (FIG. 4L) single layer microsphere system (microsphere features: quantity: 8; diameter: 0.1 mm; adjacent distance: 0.15 mm; position: middle). (FIG. 4M) single layer microsphere system (microsphere features: quantity: 8; diameter: 0.1 mm; adjacent distance: 0.25 mm; position: middle). (FIG. 4N) single layer microsphere system (microsphere features: quantity: 8; diameter: 0.1 mm; adjacent distance: 0.15 mm; position: front+middle+back). (FIG. 4O) double layer microsphere system. (FIG. 4P) bulk hydrogel system.

FIG. 5A through FIG. 5F depict GDNF volume fraction of different drug release systems under the assumption of constant growth factor mass. (FIG. 5A) comparison among single, double layer microsphere and bulk hydrogel systems. (FIG. 5B-FIG. 5E) comparison within single layer microsphere only. (FIG. 5F) comparison between three drug release systems and the combined model.

FIG. 6A through FIG. 6P depict GDNF distribution of different drug release systems under the assumption of constant growth factor mass. (FIG. 6A) single layer microsphere system (microsphere features: quantity: 8; diameter: 0.1 mm; adjacent distance: 0.2 mm; position: middle). (FIG. 6B) single layer microsphere system (microsphere features: quantity: 8; diameter: 0.1 mm; adjacent distance: 0.2 mm; position: front). (FIG. 6C) single layer microsphere system (microsphere features: quantity: 8; diameter: 0.1 mm; adjacent distance: 0.2 mm; position: back). (FIG. 6D) single layer microsphere system (microsphere features: quantity: 24; diameter: 0.1 mm; adjacent distance: 0.2 mm; position: front+middle+back). (FIG. 6E) single layer microsphere system (microsphere features: quantity: 16; diameter: 0.1 mm; adjacent distance: 0.2 mm; position: front+back). (FIG. 6F) single layer microsphere system (microsphere features: quantity: 16; diameter: 0.1 mm; adjacent distance: 0.2 mm; position: front+middle). (FIG. 6G) single layer microsphere system (microsphere features: quantity: 16; diameter: 0.1 mm; adjacent distance: 0.2 mm; position: middle+back). (FIG. 6H) single layer microsphere system (microsphere features: quantity: 6; diameter: 0.1 mm; adjacent distance: 0.2 mm; position: middle). (FIG. 6I) single layer microsphere system (microsphere features: quantity: 4; diameter: 0.1 mm; adjacent distance: 0.2 mm; position: middle). (FIG. 6J) single layer microsphere system (microsphere features: quantity: 8; diameter: 0.15 mm; adjacent distance: 0.2 mm; position: middle). (FIG. 6K) single layer microsphere system (microsphere features: quantity: 8; diameter: 0.125 mm; adjacent distance: 0.2 mm; position: middle). (FIG. 6L) single layer microsphere system (microsphere features: quantity: 8; diameter: 0.1 mm; adjacent distance: 0.15 mm; position: middle). (FIG. 6M) single layer microsphere system (microsphere features: quantity: 8; diameter: 0.1 mm; adjacent distance: 0.25 mm; position: middle). (FIG. 6N) single layer microsphere system (microsphere features: quantity: 8; diameter: 0.1 mm; adjacent distance: 0.15 mm; position: front+middle+back). (FIG. 6O) double layer microsphere system. (FIG. 6P) bulk hydrogel system.

FIG. 7A through FIG. 7G depict the results of exemplary experiments. (FIG. 7A) GDNF volume fraction and flow velocity of hydrogel film systems with different film thicknesses. (FIG. 7B, FIG. 7D and FIG. 7F) GDNF distribution of three hydrogel film systems with film thicknesses from 0.1 mm to 0.15 mm. (FIG. 7C, FIG. 7E, and FIG. 7G) flow velocity of three hydrogel film systems with film thicknesses from 0.1 mm to 0.15 mm.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of the present invention. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

“Hydrogel” refers to a water-insoluble and water-swellable cross-linked polymer that is capable of absorbing at least 3 times, or at least at least 10 times, its own weight of a liquid. “Hydrogel” can also refer to a “thermo-responsive polymer” as used herein.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

As used herein, to “alleviate” a disease, defect, disorder or condition means reducing the severity of one or more symptoms of the disease, defect, disorder or condition.

As used herein, to “treat” means reducing the frequency with which symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered.

### NGC Device

The present invention provides a nerve guidance conduit (NGC) device comprising an embedded drug-releasing substrate. The device improves nerve regeneration performance by providing long-term release of drugs, therapeutics, and/or growth factors. The device comprises intermediate cavities introduced along a length of a multi-channel NGC that act as relays to enhance regenerative cell concentrations and/or growth factor delivery to improve regeneration performance. In some embodiments, the device targets peripheral nerve injury treatments.

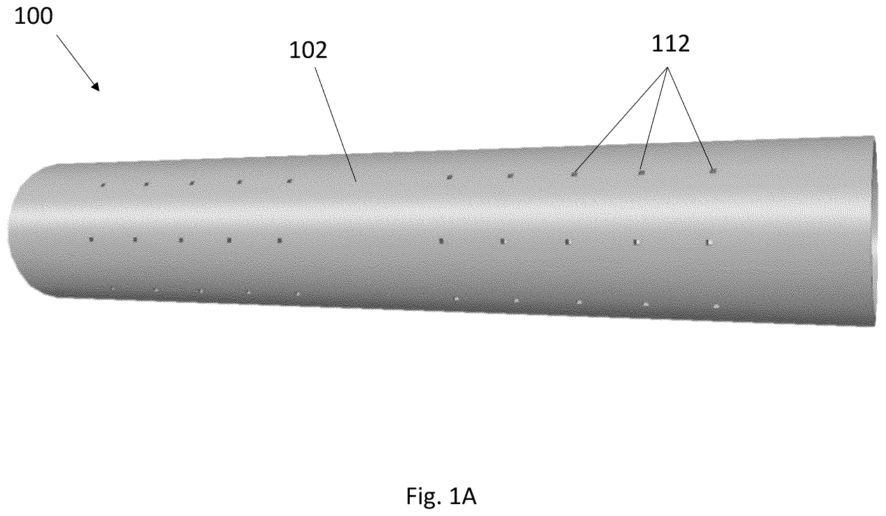

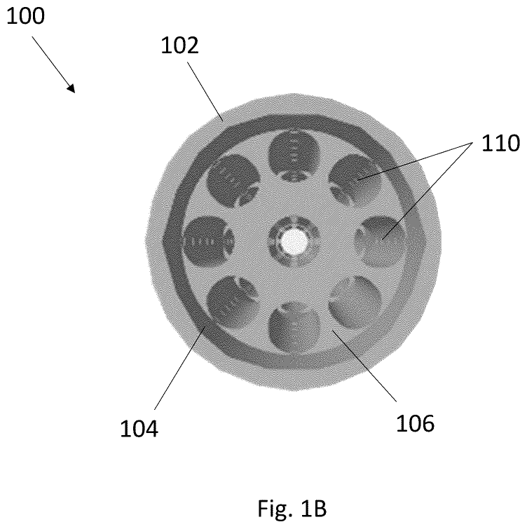

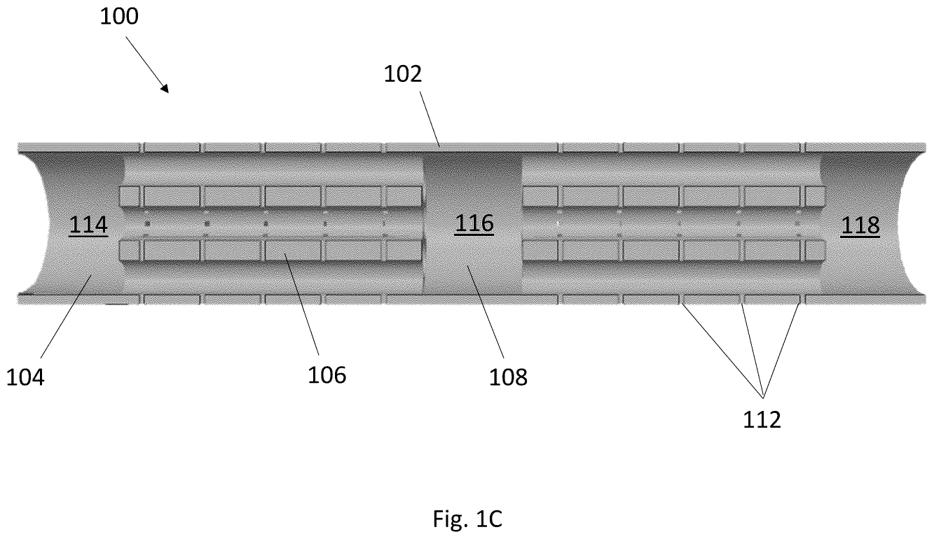

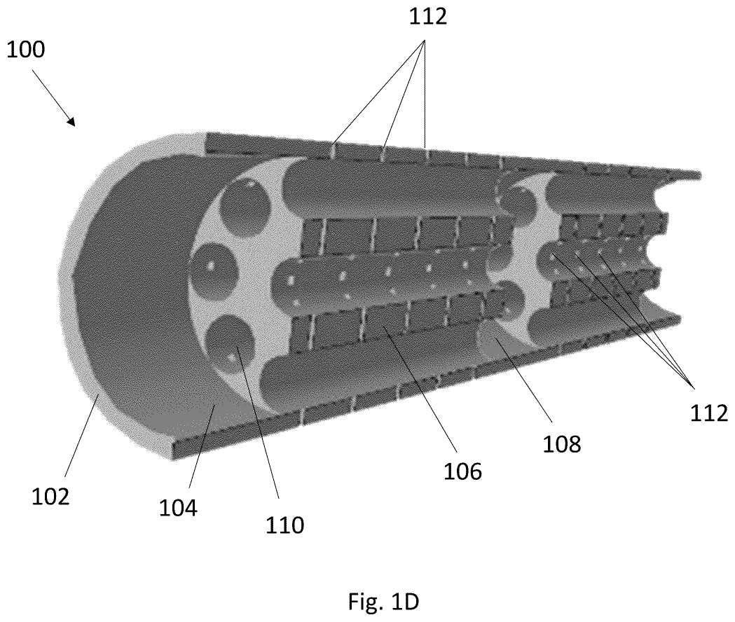

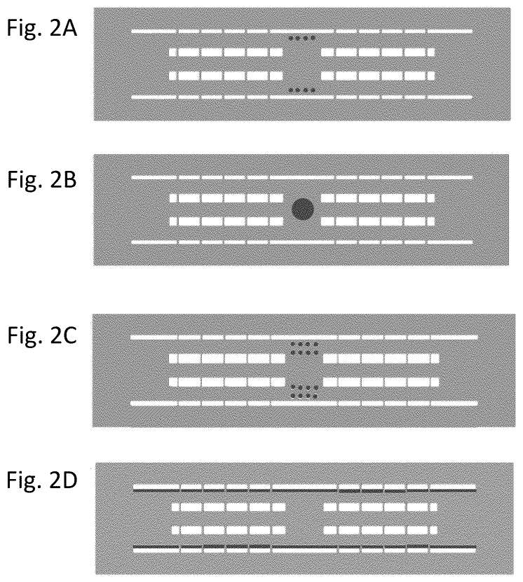

Referring now to FIG. 1A to FIG. 1D and FIG. 2A to FIG. 2D, an exemplary NGC device 100 is depicted. Device 100 comprises a tubular body having outer housing 102 with inner lumen 104 comprising a plurality of nerve-holding sections 106 and inner lumen cavities 108, such that device 100 comprises an alternating series of nerve-holding sections 106 and inner lumen cavities 108. It is to be noted that inner lumen cavities 108 may also be referred to singularly as inner lumen cavity 108, or just cavity 108. Each inner lumen cavity 108 is an empty region within inner lumen 104 that fluidly connects each nerve holding sections 106. Nerve holding section 106 is a cylindrical region with a front and back surface filling at least a portion of inner lumen 104, and comprises at least one channel 110 passing through the cylindrical region from the front surface to the back surface. Each nerve-holding section 106 comprises one or more channel 110, wherein each channel 110 is configured to receive a nerve or nerve fascicle. It should be understood that nerve-holding sections 106 within device 100 are positioned such that a plurality of channel 110 within a first nerve-holding section 106 is in alignment with a plurality of channel 110 within adjacent nerve-holding sections 106, such that a length of a nerve fascicle is receivable within several nerve-holding sections 106 without substantial kinking or bending. Device 100 further comprises a plurality of pores 112 throughout outer housing 102 and nerve-holding sections 106, such that inner lumen 104, channel 110, and inner lumen cavities 108 are fluidly connected to each other and to the exterior of outer housing 102.

In some embodiments, outer housing 102 has an exterior surface, and inner lumen 104 has an interior surface, wherein pores 112 fluidly connect the exterior surface to the interior surface. In some embodiments, pores 112 have a side surface that connects the exterior surface of outer housing 102 to the interior surface of inner lumen 104. In some embodiments, nerve-holding section 106 has a front and rear surface, wherein channel 110 fluidly connects the front and rear surfaces forming an interior surface along the length of channel 110.

Aspects of the invention relate to the materials for an NGC device. In some embodiments, device 100 comprises biocompatible and/or biodegradable materials. In some embodiments, device 100 comprises biocompatible and/or biodegradable materials that include but are not limited to poly(lactic co-glycolic acid) (PGA), poly(1-lactic-acid) (PLA), polycaprolactone (PCL), poly(ethylene glycol) diacrylate (PEGDA), gelatin methacryloyl (GelMA), polydimethylsiloxane (PDMS), collagen, chitosan, decellularized extracellular matrix (dECM), cellulose, silk, and ionic liquids. The materials could be any synthetic or natural polymer, or a composite of one or more synthetic and natural polymer(s). The material could also be made conductive by inclusion of conductive materials, nanoparticles, 2D materials such as graphene, MXenes, conductive polymers and hydrogels as conductivity plays a role in nerve regeneration. Although example materials are provided, device 100 may comprise any material appropriate for implantable medical devices, as would be known by one of ordinary level of skill in the art.

Aspects of the present invention relate to the loading of drugs, therapeutics and/or growth factors into an NGC device, such as device 100. In some embodiments, device 100 comprises at least one substrate for loading drugs, therapeutics and/or growth factors. The at least one substrate can be, for example, microparticles, microspheres, bulk hydrogel substrate, thin film hydrogel, and any combination thereof. In some embodiments, the substrate comprises a single layer of microspheres. In some embodiments, the substrate comprises a double layer of microspheres. In some embodiments, the substrate comprises a bulk hydrogel. In some embodiments, the substrate comprises a thin hydrogel film. In some embodiments, the substrate comprises PGA and/or PLA.

Device 100 is configured to receive one or more drug- or therapeutic-releasing substrates on a surface. Referring now to FIG. 2A-FIG. 2D, exemplary substrate configurations are shown, including but not limited to (FIG. 2A) a single layer of microsphere substrates, (FIG. 2B) a double layer of microsphere substrates, (FIG. 2C) a bulk hydrogel substrate, and (FIG. 2D) a thin substrate film. While the depicted microsphere substrates and bulk hydrogel substrates are depicted as located within inner lumen cavity 108 adjacent to nerve-holding section 106, it should be understood that the substrates can be located in any position along outer housing 102 and inner lumen 104. In some embodiments device 100 comprises locations for positioning the at least one substrate that may be loaded with drugs, therapeutics and/or growth factors. Now referring to FIG. 1C, shown are inner lumen substrate locations: front 114, middle 116, and back 118, wherein drugs, therapeutics and/or growth factors may be loaded. It is to be noted that the at least one substrate may be located in some or all of the inner lumen substrate locations found in device 100.

Aspects of the present invention relates to coatings on the surfaces of an NGC device. In some embodiments, device 100 comprises coatings on some or all of the surfaces. For example, coatings may be present on the exterior surface of outer housing 102, the interior surface of inner lumen 104, the side surface of pores 112, the front and/or back surface of nerve-holding sections 106, the surface of inner lumen cavities 108, and/or the interior surface of channel 110. In some embodiments, device 100 comprises a coating that responds to external stimuli such as magnetic field, electric field, or temperature. In some embodiments, the coating may comprise conductive materials, ferromagnetic materials and/or thermoelectric materials. In some embodiments, the coating may comprise hydrogel material. In some embodiments, the coating comprises biocompatible and/or biodegradable materials that include but are not limited to poly(lactic co-glycolic acid) (PGA), poly(1-lactic-acid) (PLA), polycaprolactone (PCL), poly(ethylene glycol) diacrylate (PEGDA), gelatin methacryloyl (GelMA), polydimethylsiloxane (PDMS), collagen, chitosan, decellularized extracellular matrix (dECM), cellulose, silk, and ionic liquids. The coating materials could be any synthetic or natural polymer, or a composite of one or more synthetic and natural polymer(s). The coating material could also be made conductive by inclusion of conductive materials, nanoparticles, 2D materials such as graphene, MXenes, conductive polymers and hydrogels as conductivity plays a role in nerve regeneration.

In some embodiments, the at least one substrate of device 100 comprises at least one microparticle. Microparticles are generally understood by persons having skill in the art to refers to small particles which behave as a whole unit in terms of their transport and properties, and which typically exhibit an average particle size diameter (determined, for example, by a microscopy, electrozone sensing, or laser diffraction technique) in the range of about 0.1 to 10 μm or greater. Terms that may be used synonymously with microparticle include but are not limited to: nanoparticle, micro- and nanobubble, micelle, micro- and nanosphere, micro- and nanocapsule, micro- and nanobead, micro- and nanosome, and the like. Microparticles may comprise any structure suitable for the delivery of a desired therapeutic. For example, a microparticle may comprise a vesicle-like structure composed of a fluid core encased in a membrane comprising a lipid bilayer. Alternatively, a microparticle may comprise a hydrophilic shell and a hydrophobic core. A microparticle may also comprise one or more solid cores, or a distribution of solid or fluid deposits within a matrix.

The microparticles may be uncoated or coated to impart a charge or to alter lipophilicity. Microparticles may have a uniform shape, such as a sphere (e.g. a microsphere). Microparticles may also be irregular, crystalline, semi-crystalline, or amorphous. A single type of microparticle may be used, or mixtures of different types of microparticles may be used. If a mixture of microparticles is used they may be homogeneously or non-homogeneously distributed. In various aspects, the microparticle is biodegradable or non-biodegradable, or in a plurality of microparticles, combinations of biodegradable and non-biodegradable cores are contemplated.

In some embodiments, the microparticles comprise a polymer. Non-limiting examples of suitable polymers include but are not limited to PLGA, PLA, PGA, PCL, PLL, cellulose, poly(ethylene-co-vinyl acetate), polystyrene, polypropylene, dendrimer-based polymers, polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polysebacates, poly(glycerolsebacates), poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly(β-amino acids) (either homopolymers or random copolymers), poly(n-vinyl pyrrolidone) polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. For example, in some embodiments, the PLGA comprises any PLGA known in the art, including, but not limited to, 99:1 PLGA, 95:5 PLGA, 90:10 PLGA, 85:15 PLGA, 80:20 PLGA, 75:25 PLGA, 70:30 PLGA, 65:35 PLGA, 60:40 PLGA, 55:45 PLGA, 50:50 PLGA, 45:55 PLGA, 40:60 PLGA, 35:65 PLGA, 30:70 PLGA, 25:75 PLGA, 20:80 PLGA, 15:85 PLGA, 10:90 PLGA, 5:95 PLGA, and/or 1:99 PLGA.

In some aspects, the substrate comprises a hydrogel. Hydrogels can generally absorb a great deal of fluid and, at equilibrium, typically are composed of 60-90% fluid and only 10-30% polymer. In a preferred embodiment, the water content of hydrogel is about 70-80%. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). The hydrogels may be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. (see.: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of hydrogels based on chemical or physical crosslinking synthetic polymers include but are not limited to (meth)acrylate-oligolactide-PEO-oligolactide-(meth)acrylate, poly(ethylene glycol) diacrylate (PEGDA), poly(ethylene glycol) (PEO), poly(propylene glycol) (PPO), PEO—PPO-PEO copolymers (Pluronics), poly(phosphazene), poly(methacrylates), poly(N-vinylpyrrolidone), PL(G)A-PEO-PL(G)A copolymers, poly(ethylene imine), etc. (see A. S Hoffman, 2002, Adv. Drug Del. Rev, 43, 3-12).

In one embodiment, the hydrogel comprises at least one biopolymer. In other embodiments, the hydrogel scaffold further comprises at least two biopolymers. In yet other embodiments, the hydrogel scaffold comprises at least one biopolymer and at least one synthetic polymer.

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels may also be made degradable in vivo by incorporating PLA, PLGA or PGA polymers. Moreover, hydrogels may be modified with fibronectin, laminin, vitronectin, or, for example, RGD for surface modification, which may promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes may influence strength, elasticity, and degradation properties of the instant hydrogels (Nguyen and West, 2002, Biomaterials 23(22):4307-14; Ifkovits and Burdick, 2007, Tissue Eng. 13(10):2369-85).

Hydrogels may also be modified with functional groups for covalently attaching a variety of proteins or compounds such as therapeutic agents. It is contemplated that linkage of the therapeutic agent to the matrix may be via a protease sensitive linker or other biodegradable linkage.

In certain embodiments, one or more multifunctional cross-linking agents may be utilized as reactive moieties that covalently link biopolymers or synthetic polymers. Such bifunctional cross-linking agents may include glutaraldehyde, genipin, epoxides (e.g., bis-oxiranes), oxidized dextran, p-azidobenzoyl hydrazide, N-[α-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NHS) and other bifunctional cross-linking reagents known to those skilled in the art. It should be appreciated by those in skilled in the art that the mechanical properties of the hydrogel are greatly influenced by the cross-linking time and the amount of cross-linking agents.

In another embodiment utilizing a cross-linking agent, polyacrylated materials, such as ethoxylated (20) trimethylpropane triacrylate, may be used as a non-specific photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as eosin Y, catalytic agents, such as 1-vinyl-2-pyrrolidinone, and triethanolamine. Continuous exposure of this reactive mixture to long-wavelength light (>498 nm) would produce a cross-linked hydrogel network.

In one embodiment, the hydrogel comprises a UV sensitive curing agent which initiates hydrogel polymerization. For example, in one embodiment, a hydrogel comprises the photoinitiator 4-(2-hydroxyethoxy)phenyl-(2-hydroxy-2-propyl)ketone. In one embodiment, polymerization is induced by 4-(2-hydroxyethoxy)phenyl-(2-hydroxy-2-propyl)ketone upon application of UV light. Other examples of UV sensitive curing agents include 2-hydroxy-2-methyl-1-phenylpropan-2-one, 4-(2-hydroxyethoxy)phenyl (2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

The stabilized cross-linked hydrogel matrix of the present invention may be further stabilized and enhanced through the addition of one or more enhancing agents. By “enhancing agent” or “stabilizing agent” is intended any compound added to the hydrogel matrix, in addition to the high molecular weight components, that enhances the hydrogel matrix by providing further stability or functional advantages. Suitable enhancing agents, which are admixed with the high molecular weight components and dispersed within the hydrogel matrix, include many of the additives described earlier in connection with the thermo-reversible matrix discussed above. The enhancing agent may include any compound, especially polar compounds, that, when incorporated into the cross-linked hydrogel matrix, enhance the hydrogel matrix by providing further stability or functional advantages.

Exemplary enhancing agents for use with the stabilized cross-linked hydrogel matrix include polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine, and histidine. The preferred polar amino acids are L-cysteine, L-glutamic acid, L-lysine, and L-arginine. Suitable concentrations of each particular preferred enhancing agent are the same as noted above in connection with the thermo-reversible hydrogel matrix. Polar amino acids, EDTA, and mixtures thereof, are preferred enhancing agents. The enhancing agents may be added to the matrix composition before or during the crosslinking of the high molecular weight components.

Aspects of the present invention relate to drugs, therapeutics, and the like, loaded into an NGC device for stimulating growth of damaged nerves. Contemplated drugs or therapeutics include but are not limited to growth factors, neurotrophic factors, cell adhesion molecules, proteins, peptides, small molecules, nucleic acid molecules, cytokines, stem cells, Schwann cells, upregulators of regeneration-associated genes, conductive biocompatible materials, including but are not limited to polypyrrole (PPy), poly(3,4-ethylenedioxythiophene) polystyrene sulfonate (PEDOT:PSS), graphene, carbon nanotubes, metal nanoparticles, ionic liquids, and the like.

Exemplary growth factors or neurotrophic factors that can be embedded and released from the substrate include but are not limited to, glial cell derived neurotrophic factor (GDNF), nerve growth factor (NGF), epidermal growth factor (EGF), vascular endothelial growth factor (VEGF), fibroblast growth factor (FGF), ciliary neurotrophic factor (CNTF), platelet derived growth factor (PDGF), brain derived neurotrophic factor (BDNF), basic fibroblast growth factor (bFGF), neurotrophin 3 (NT-3), and neurotrophin 4 (NT-4), insulin-like growth factor 2 (IGF-2), and the like.

Aspects of the present invention relate to the dimensions and configurations of an NGC device. In some embodiments, outer housing 102 has a length and a diameter. In some embodiments, outer housing 102 has a length of about 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.5 mm. In some embodiments, outer housing 102 has a length of at least 3.0 mm. For example, in some embodiments, the length of outer housing 102 is 10.0 mm.

In some embodiments, outer housing 102 has a diameter of about 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, or about 3.0 mm. In some embodiments, outer housing 102 has a diameter of at least 1.0 mm. For example, in some embodiments the diameter of device 100 is 2.0 mm. In some embodiments, outer housing 102 has a diameter of at least 0.1 mm.

In some embodiments, outer housing 102 has a wall thickness. In some embodiments, outer housing 102 has a wall thickness of about 50 μm, 100 μm, 150 μm, 200 μm, 250 μm, 300 μm, 350 μm, 400 μm, or about 450 μm. In some embodiments, outer housing 102 has a wall thickness of at least 25 μm.

In some embodiments, nerve holding section 106 has a quantity of channel 110. In some embodiments, nerve holding section 106 has about 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 channel 110. For example, in some embodiments, nerve holding section 106 has 9 channel 110.

In some embodiments, channel 110 has at least one diameter of about 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or about 1000 μm. For example, in some embodiments, channel 110 has a diameter of 340 μm. It should be noted that channel 110 may comprise more than one diameter, and in some embodiments, the channels 110 have different diameters. In some embodiments, channel 110 has diameters ranging from 50 μm to 1000 μm.

In some embodiments, inner lumen cavity 108 has a length. In some embodiments, inner lumen cavity 108 has a length of about 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, or about 3.0 mm. In some embodiments, inner lumen cavity 108 has a length of at least 1.5 mm. For example, in some embodiments, the length of inner lumen cavity 108 is 5.0 mm.

In some embodiments, pores 112 comprise at least one diameter. In some embodiments, pores 112 have a diameter of about 1 μm, 2 μm, 3 μm, 4 μm, 5 μm, 6 μm, 7 μm, 8 μm, 9 μm or about 10 μm. For example, in some embodiments, pores 112 have a diameter of 3 μm. In some embodiments, pores 112 have a diameter of about 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm or about 250 μm. For example, in some embodiments, pores 112 have a diameter of 150 μm. In some embodiments, pores have a diameter ranging from 1 μm to 10 μm. It should be noted that pores 112 may comprise more than one diameter. For example, pores 112 in outer housing 102 may have a different diameter from pores 112 in nerve holding section 106.

In some embodiments, device 100 has a porosity. In some embodiments, device 100 has a porosity of about 0%, 5%, 10%, 15%, 20%, 25%, 30%, 35%, 40%, 45%, 50%, 55%, 60%, 65%, 70%, 75%, 80%, 85%, 90%, 95%, or about 100%. For example, in some embodiments, device 100 has a porosity of 40%. In some embodiments, the porosity of the outer housing 102 ranges from 10% to 90%.

In some embodiments, device 100 comprises at least one substrate comprising microparticles configured as a drug, therapeutic, or growth factor-releasing substrate. In some embodiments, the microparticles are configured as a single layer or multiple layers of microparticles. In some embodiments, the microparticles may be described as having a quantity, diameter, location and spacing between adjacent microparticle.

In some embodiments, the at least one substrate comprises about 5, 10, 15, 20, 25, 30, 35, 40, 45, 50, 55, 60, 65, 70, 75, 80, 85, 90, 95, or about 100 microparticles. In some embodiments, the at least one substrate comprises at least 100 microparticles. For example, in some embodiments, the at least one substrate has 150 microparticles.

In some embodiments, the microparticles are microspheres with at least one diameter of about 25 μm, 50 μm, 75 μm, 100 μm, 125 μm, 150 μm, 175 μm, 200 μm, 225 μm, 250 μm, 275 μm, or about 300 μm. For example, in some embodiments, the microspheres have a diameter of 125 μm. In some embodiments, the microspheres have different diameters. For example, microspheres may a different diameter based upon their position within inner lumen 104.

In some embodiments, a microparticle is spaced about 0.05 mm, 0.075 mm, 0.1 mm, 0.125 mm, 0.15 mm, 0.175 mm, 0.2 mm, 0.225 mm, 0.25 mm, 0.275 mm, 0.3 mm, 0.325 mm, 0.35 mm, 0.375 mm, or about 0.4 mm from the adjacent microparticle (also referred to herein as “adjacent distance”). In some embodiments, the adjacent distance ranges from 0.01 mm to 2 mm. For example, in some embodiments, each microparticles is spaced about 0.1 mm away from the adjacent microparticle.

Now referring to FIG. 1C, in some embodiments, the microparticles are placed in front 114, middle 116 and back 118 locations within inner lumen 104 and/or various combinations of placements thereof. For example, in some embodiments, an exemplary substrate for device 100 comprises a double layer of 8 microspheres with 0.1 mm diameter, spaced 0.15 mm from adjacent microsphere, and front+middle+back inner lumen cavity placement. Although this example is provided, any variation of microspheres and/or configurations may form a microsphere substrate for device 100 according to aspects of the present invention. According to the disclosed CFD simulation analysis, under the assumptions of constant simulation time and constant growth factor mass, the double-layer microsphere system which is placed in the middle inner lumen cavity achieves the best growth factor release performance. While this is just an example, the drug release system can be optimized in a number of different ways to achieve the best physiological response and nerve regeneration.

In some embodiments, device 100 comprises at least one bulk hydrogel substrate. In some embodiments, the at least one bulk hydrogel substrate is configured as a plug or disc, having a diameter and a thickness, that fills a portion of inner lumen cavities 108. Although example shapes for the bulk hydrogel substate are provided, it should be understood that bulk hydrogel substrate may be any shape. In some embodiments, the at least one bulk hydrogel substrate has a diameter of about 100 μm, 200 μm, 300 μm, 400 μm, 500 μm, 600 μm, 700 μm, 800 μm, 900 μm, or about 1000 μm.

In some embodiments, the bulk hydrogel has a diameter of at least 100 μm. In some embodiments, the at least one bulk hydrogel may have different diameters. For example, the diameter of the bulk hydrogel substrate may vary based on the position of the substrate in inner lumen 104.

In some embodiments, the bulk hydrogel substrate has a thickness of about 100 μm, 200 μm, 300 μm, 400 μm, 500 μm, 600 μm, 700 μm, 800 μm, 900 μm, or about 1000 μm. In some embodiments, the bulk hydrogel has a thickness of at least 100 μm. The bulk hydrogel substrate can be varied with respect to the diameter and length of outer housing 102, inner lumen 104, and inner lumen cavities 108.

In some embodiments, the hydrogel film substrate has a thickness. In some embodiments, the hydrogel film substrate has a thickness of about 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm or about 200 μm. In some embodiments, the hydrogel film substrate has a thickness of about 0.1 mm, 0.2 mm, 0.3 mm, 0.4 mm, 0.5 mm, 0.6 mm, 0.7 mm, 0.8 mm, 0.9 mm or about 1.0 mm. In some embodiments, the hydrogel film has a thickness between 0.1 mm and 0.2 mm.

In some embodiments, the duration of the drug, therapeutic, and/or growth factor release profile is about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days 49 days, 50 days, 51 days, 52 days, 53 days, 54 days, 55 days, 56 days, 57 days, 58 days, 59 days, or about 60 days.

### Methods of Use

A further embodiment of the invention refers to the nerve guidance conduit as defined in the previous paragraphs for use in a medical treatment. It is a further aspect of the present invention the nerve guidance conduit as defined in preceding paragraphs for treating a nerve injury in a subject, such as animals and humans. In certain embodiments, the nerve injury includes peripheral nerve injury. In a further aspect of the present invention the nerve guidance conduit is for use in reconstructing nerve gaps. In some embodiments, the nerve guidance conduit is for use in the treatment of spinal cord injury.

Aspects of the present invention relate to a method for regenerating a damaged, injured, or severed nerve in a subject in need thereof, comprising the steps of: placing the implantable nerve guidance conduit as defined in previous paragraphs, at the site of neuronal injury so as to regenerate the nerve. As described above, in certain embodiments, the nerve guidance conduit comprises a tubular outer housing having an inner lumen; and a plurality of nerve-holding sections positioned within the inner lumen of the housing, each nerve-holding section comprising one or more channels each configured to receive a nerve; and the inner lumen of the housing is divided into an alternating series of nerve-holding sections and inner lumen cavities, wherein the inner lumen comprises at least one drug or therapeutic-releasing substrate.

Another embodiment relates to a method of treating nerve disorder comprising placing a nerve guide conduit as defined in the previous paragraphs in a damaged, injured or severed nerve pathway. As described above, in certain embodiments, the nerve guidance conduit comprising a tubular outer housing having an inner lumen; and a plurality of nerve-holding sections positioned within the inner lumen of the housing, each nerve-holding section comprising one or more channels each configured to receive a nerve; and the inner lumen of the housing is divided into an alternating series of nerve-holding sections and inner lumen cavities, wherein the inner lumen comprises at least one drug or therapeutic-releasing substrate. The whole NGC structure or part of it can be made conductive by inclusion of conductive materials and external electrical or magnetic fields can be used to enhance the rate of nerve regeneration. The drug-release system can be made smart or stimuli-responsive where any external means such as magnetic field, electric field, or temperature could be used to trigger the drug release inside the body after the device 100 has been transplanted into the body.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

Peripheral nerve injuries can result from either systemic disease (e.g., diabetes, Guilain-Barre syndrome, carpal tunnel syndrome) or localized damage (e.g., trauma, sports-related stretching/compression, tumor extirpation). Peripheral nerve injuries can be classified into five stages with increasing severity, starting from a self-restorable local conduction block to a complete transection of the nerve. It is hard for the nerve to self-regenerate when it experiences a complete transection, therefore an external treatment is required to promote the nerve regeneration. Currently, the treatments used can be classified into three major groups: direct coaptation, grafts and nerve guidance conduits (NGC). Small nerve gaps (<8 mm) can be simply repaired by direct coaptation, which is the most frequently used clinical treatment owning to its short operation time and complete consistency (e.g., axon size, number, distribution) between both sides of the nerves [Cinteza, D., et al., Peripheral Nerve Regeneration—an Appraisal of the Current Treatment Options. Maedica, 2015. 10(1): p. 65-68; Sedaghati, T., G. Jell, and A. M. Seifalian, Chapter 57-Nerve Regeneration and Bioengineering, in Regenerative Medicine Applications in Organ Transplantation, G. Orlando, et al., Editors. 2014, Academic Press: Boston. p. 799-810; Dietzmeyer, N., et al., Modification of tubular chitosan-based peripheral nerve implants: Applications for simple or more complex approaches. Neural Regeneration Research, 2020. 15(8): p. 1421-1431 DOI: 10.4103/1673-5374.271668]. For nerve gaps larger than 8 mm, injured nerve can break due to insufficient elasticity and the exceeded tension can damage the blood flow and impede the nerve regeneration [Hood, B., H. B. Levene, and A. D. Levi, Transplantation of autologous Schwann cells for the repair of segmental peripheral nerve defects. Neurosurgical Focus, 2009. 26(2): p. 1-9 DOI: 10.3171/FOC.2009.26.2.E4]. Therefore, grafts are more suitable for long-gap nerve injuries rather than direct coaptation. Grafts can be divided into allograft and autograft. Autograft involves harvesting a section of the nerve from the patient and then transplanting it directly to the injury site. Sural nerve is commonly used as the autograft nerve because of the fast harvesting operation, sufficient length and fascicular groups [Cinal, H., et al., A new method to harvest the sural nerve graft. Eurasian Journal of Medicine, 2020. 52(1): p. 12-15 DOI: 10.5152/eurasianjmed.2019.19102]. Although autograft suffers from donor site morbidity, considering its excellent regeneration performance, it remains the first choice for long-gap nerve injuries (>4 cm) and is treated as the gold standard among all the clinical methods [Zarrintaj, P., et al., Conductive biomaterials as nerve conduits: Recent advances and future challenges. Applied Materials Today, 2020. 20 DOI: 10.1016/j.apmt.2020.100784]. The widespread applications of allograft are limited by donor availability and immune rejection reaction, resulting in the inferior status of allografts compared with autografts [Gupta, S., et al., Human skin allograft: Is it a viable option in management of burn patients? Journal of Cutaneous and Aesthetic Surgery, 2019. 12(2): p. 132-135 DOI: 10.4103/JCAS.JCAS_83_18].

Thus, NGC is developed to overcome the limitations posed by direct coaptation and graft treatments. Normally NGC is a tubular structure acting as a bridge to connect both proximal and distal sides of the injured nerve. Owning to the advantages of patient specific customization, availability & synthetic materials and cell-laden bioinks, NGC is considered of a potential alternative for clinical treatment [Zhang, S., et al., Computational Design and Optimization of Nerve Guidance Conduits for Improved Mechanical Properties and Permeability. Journal of Biomechanical Engineering, 2019. 141(5) DOI: 10.1115/1.4043036; Spencer, A. R., et al., Bioprinting of a Cell-Laden Conductive Hydrogel Composite. ACS Applied Materials and Interfaces, 2019. 11(34): p. 30518-30533 DOI: 10.1021/acsami.9b07353]. However, current commercially available NGCs mostly target medium nerve gaps (<3 cm) with poorer regeneration performance compared to autograft [Fadia, N. B., et al., Long-gap peripheral nerve repair through sustained release of a neurotrophic factor in nonhuman primates. Science Translational Medicine, 2020. 12(527) DOI: 10.1126/scitranslmed.aav7753]. Since NGC alone produces unsatisfactory results, changes are required to create a more biomimetic environment by integrating growth factors and cells with the NGC. Growth factors are up-regulated immediately after the peripheral nerve injuries followed by a gradient decrease after long-term denervation [Xu, P., et al., Nerve injury induces glial cell line-derived neurotrophic factor (GDNF) expression in schwann cells through purinergic signaling and the PKC-PKD pathway. GLIA, 2013. 61(7): p. 1029-1040 DOI: 10.1002/glia.22491; Höke, A., et al., A decline in glial cell-line-derived neurotrophic factor expression is associated with impaired regeneration after long-term Schwann cell denervation. Experimental Neurology, 2002. 173(1): p. 77-85 DOI: 10.1006/exnr.2001.7826]. Packing growth factors with NGC can maintain a proper growth factor concentration for a long time, supporting cell proliferation and cell survival [Troullinaki, M., et al., Nerve growth factor regulates endothelial cell survival and pathological retinal angiogenesis. Journal of Cellular and Molecular Medicine, 2019. 23(4): p. 2362-2371 DOI: https://doi.org/10.1111/jcmm.14002; Vijayavenkataraman, S., Nerve guide conduits for peripheral nerve injury repair: A review on design, materials and fabrication methods. Acta Biomaterialia, 2020. 106: p. 54-69 DOI: 10.1016/j.actbio.2020.02.003]. Cells cultured in cell-laden NGC can proliferate and migrate to both sides of the injured nerve, leading to faster nerve regeneration. By providing a proper electrical stimulus, regeneration performance can be further improved by regulating cell behaviors (e.g., cell differentiation, cell migration). However, FDA approval is required for the clinical translation of NGC. [Cinteza, D., et al., Peripheral Nerve Regeneration—an Appraisal of the Current Treatment Options. Maedica, 2015. 10(1): p. 65-68] Most commercially FDA approved NGCs are made from natural material with superior biocompatibility and biodegradability [Arslantunali, D., et al., Peripheral nerve conduits: Technology update. Medical Devices: Evidence and Research, 2014. 7: p. 405-424 DOI: 10.2147/MDER.S59124]. Compared with growth factor embedded NGC, cell-laden NGC requires a much longer time to receive the FDA approval because the mechanism and potential risk of cell differentiation is not fully understood [Zhou, X., E. Quann, and G. I. Gallicano, Differentiation of nonbeating embryonic stem cells into beating cardiomyocytes is dependent on downregulation of PKCβ and ζ in concert with upregulation of PKCs. Developmental Biology, 2003. 255(2): p. 407-422 DOI: 10.1016/S0012-1606(02) 00080-5]. Therefore, growth factor embedded NGC takes the dominant position benefiting from the simple clinical translation procedure.

Sustained release of growth factors can be achieved by combining growth factors with biodegradable microspheres and hydrogels, thus promoting nerve regeneration in large peripheral nerve gaps [Kokai, L. E., et al., Sustained growth factor delivery promotes axonal regeneration in long gap peripheral nerve repair. Tissue Engineering-Part A, 2011. 17(9-10): p. 1263-1275 DOI: 10.1089/ten.tea.2010.0507; Silva, A. K. A., et al., Growth factor delivery approaches in hydrogels. Biomacromolecules, 2009. 10(1): p. 9-18 DOI: 10.1021/bm801103c]. A well-established microsphere delivery system is made of poly(lactic-co-glycolic acid), poly(l-lactic acid) and growth factors [Kokai, L. E., A. M. Ghaznavi, and K. G. Marra, Incorporation of double-walled microspheres into polymer nerve guides for the sustained delivery of glial cell line-derived neurotrophic factor. Biomaterials, 2010. 31(8): p. 2313-2322 DOI: 10.1016/j.biomaterials.2009.11.075]. Those microspheres were embedded in the inner layer of a single channel NGC, which was then implanted into a 5 cm nerve defect in a rhesus macaque model for over a year [Fadia, N. B., et al., Long-gap peripheral nerve repair through sustained release of a neurotrophic factor in nonhuman primates. Science Translational Medicine, 2020. 12(527) DOI: 10.1126/scitranslmed.aav7753]. The results showed a sustained release of growth factor over 50 days and identical or even superior regeneration performance compared with autograft. As hydrogels are highly hydrophilic, different strategies of growth factor release need to be applied in an effort to maintain a sustained release other than a rapid burst release. Depending on the immobilization mechanisms, the strategies can be classified into physical encapsulation, covalent conjunctions and extra cellular matrix-inspired immobilization [Wang, Z., et al., Novel biomaterial strategies for controlled growth factor delivery for biomedical applications. NPG Asia Materials, 2017. 9(10): p. e435-e435 DOI: 10.1038/am.2017.171]. Growth factor release profiles vary from couple days up to 50 days based on the structure pattern and strategies applied [Murphy, W. L., et al., Sustained release of vascular endothelial growth factor from mineralized poly(lactide-co-glycolide) scaffolds for tissue engineering. Biomaterials, 2000. 21(24): p. 2521-2527 DOI: 10.1016/S0142-9612(00) 00120-4; Cho, H. J., et al., Effective immobilization of BMP-2 mediated by polydopamine coating on biodegradable nanofibers for enhanced in vivo bone formation. ACS Applied Materials and Interfaces, 2014. 6(14): p. 11225-11235 DOI: 10.1021/am501391z]. To achieve a uniform nerve regeneration rate across the injury site, it is important to evenly distribute growth factors within the NGC. Given the tiny size of NGC (2-9 mm in diameter, 200 μm wall thickness), the drug release system needs to be optimized to maximize the growth factor concentration under the limited size of microspheres and hydrogels.

In the disclosed study, a 2D multichannel NGC was created using computational fluid dynamics (CFD) simulation to examine the transporting efficiency of various growth factor delivery systems, which are single layer microspheres, double layer microspheres, bulk hydrogels and hydrogel films. Growth factor release profile is derived from a published resource [Kokai, L. E., A. M. Ghaznavi, and K. G. Marra, Incorporation of double-walled microspheres into polymer nerve guides for the sustained delivery of glial cell line-derived neurotrophic factor. Biomaterials, 2010. 31(8): p. 2313-2322 DOI: 10.1016/j.biomaterials.2009.11.075], then the growth factor volume fraction is calculated and used as a criterion to examine the growth factor release performance. For the single layer microsphere system, microsphere features, including microspheres quantity, diameter, location and adjacent distance, can be further modified. The aim of this study is to evaluate the magnitude and distribution of growth factor under different delivery systems by giving either a fixed release time or a constant growth factor releases mass. Finally, the preferred growth factor delivery system is selected for optimal growth factor release performance in the multichannel NGC.

The materials and methods are now described.

### 2D Multichannel NGC Model

To achieve a better biomimetic structure, multichannel NGC with 40% porosity was chosen and constructed using ANSYS DesignModeler Geometry (Version 2020 R2) [Koffler, J., et al., Biomimetic 3D-printed scaffolds for spinal cord injury repair. Nature Medicine, 2019. 25(2): p. 263-269 DOI: 10.1038/s41591-018-0296-z]. Each channel corresponds to a single nerve fascicle, which allows parallel nerve regeneration, thereby improving the nerve regeneration efficiency. FIG. 1C shows the 3D multichannel NGC models with three internal locations (front, middle and back) available for loading growth factor systems. Based on previous literature, channel diameter and quantities of multichannel NGC can be varied from 200 μm to 660 μm and 4 to 30, respectively [Koffler, J., et al., Biomimetic 3D-printed scaffolds for spinal cord injury repair. Nature Medicine, 2019. 25(2): p. 263-269 DOI: 10.1038/s41591-018-0296-z; Yao, L., et al., Improved axonal regeneration of transected spinal cord mediated by multichannel collagen conduits functionalized with neurotrophin-3 gene. Gene Therapy, 2013. 20(12): p. 1149-1157 DOI: 10.1038/gt.2013.42; Pawelec, K. M., et al., Microstructure and in vivo characterization of multi-channel nerve guidance scaffolds. Biomedical Materials (Bristol), 2018. 13(4) DOI: 10.1088/1748-605X/aaad85; Zhao, X., et al., Bioinspired multichannel nerve guidance conduit based on shape memory nanofibers for potential application in peripheral nerve repair. ACS Nano, 2020. 14(10): p. 12579-12595 DOI: 10.1021/acsnano.0c03570; Yao, L., et al., Multichanneled collagen conduits for peripheral nerve regeneration: Design, fabrication, and characterization. Tissue Engineering-Part C: Methods, 2010. 16(6): p. 1585-1596 DOI: 10.1089/ten.tec.2010.0152]. Furthermore, the diameter of human sciatic nerve ranges from 2 mm to 9 mm depending on the location [Gustafson, K. J., et al., Human distal sciatic nerve fascicular anatomy: Implications for ankle control using nerve-cuff electrodes. Journal of Rehabilitation Research and Development, 2012. 49(2): p. 309-322 DOI: 10.1682/JRRD.2010.10.0201]. Therefore, in order to ensure the rationality of the parameters and maintain a suitable porosity (40%), structural features of multichannel NGC including NGC diameter (2 mm), channel diameter (340 μm) and channel quantity (9) were selected in this study. To reduce the simulation complexity, 2D multichannel NGC was chosen and acted as a representative to evaluate the performance of different drug release systems in multichannel NGC.

### Drug Release Systems

Four types of drug release systems were constructed and embedded in the 2D multichannel NGC, namely single layer microsphere system, double layer microsphere system, growth factor embedded bulk hydrogel and hydrogel film, as shown in FIG. 2. Microspheres can be fabricated by oil-in-oil emulsion following a 10 minutes centrifugation, and the sizes of microsphere were mainly between 100 μm and 200 μm [Fadia, N. B., et al., Long-gap peripheral nerve repair through sustained release of a neurotrophic factor in nonhuman primates. Science Translational Medicine, 2020. 12(527) DOI: 10.1126/scitranslmed.aav7753; Kokai, L. E., A. M. Ghaznavi, and K. G. Marra, Incorporation of double-walled microspheres into polymer nerve guides for the sustained delivery of glial cell line-derived neurotrophic factor. Biomaterials, 2010. 31(8): p. 2313-2322 DOI: 10.1016/j.biomaterials.2009.11.075]. For both single- and double-layer microsphere systems, the microsphere had diameters of 100 μm and were placed symmetrically on the upper and lower surfaces of the middle cavity. Microsphere quantities were 8 and 16 for both systems respectively, due to the constraint of cavity length. For the single layer microsphere system, microsphere features including diameter (100-150 μm), quantity (4-8), adjacent distance (0.15-0.25 mm) and locations (front, middle, back) were further modified to examine the growth factor releasing performance. The default setting of single layer microsphere system is 8 microspheres, 0.1 mm diameter, 0.2 mm adjacent distance and middle cavity placement, which performs as a standard for all variations of microsphere features. The development of injectable hydrogel enables rapid sol-gel transition time, allowing the formation of hydrogel directly in the middle cavity without breaking the structure into two parts [Xu, H., et al., Preparation and characterization of injectable chitosan-hyaluronic acid hydrogels for nerve growth factor sustained release. Journal of Bioactive and Compatible Polymers, 2017. 32(2): p. 146-162 DOI: 10.1177/0883911516662068]. Considering the inherent biodegradability of hydrogels, the bulk hydrogel diameter of 500 μm was determined, accounting for half the size of the middle cavity. Apart from bulk hydrogels in the middle cavity, hydrogel can also be coated on the inner surface as a thin layer from proximal side to distal side of the multichannel NGC. Growth factor embedded hydrogel film has been widely used in skin regeneration and wound healing with hydrogel thickness ranging from 30 μm to 1 mm depending on different hydrogel materials [Wang, P., et al., Controlled Growth Factor Release in 3D-Printed Hydrogels. Advanced Healthcare Materials, 2020. 9(15) DOI: 10.1002/adhm.201900977; Trujillo, S., et al., Engineered 3D hydrogels with full-length fibronectin that sequester and present growth factors. Biomaterials, 2020. 252: p. 120104 DOI: 2020.120104; Kim, H., et al., Hyaluronate-Epidermal Growth Factor Conjugate for Skin Wound Healing and Regeneration. Biomacromolecules, 2016. 17(11): p. 3694-3705 DOI: 10.1021/acs.biomac.6b01216; Hong, J. P., et al., The effect of continuous release of recombinant human epidermal growth factor (rh-EGF) in chitosan film on full thickness excisional porcine wounds. Annals of Plastic Surgery, 2008. 61(4): p. 457-462 DOI: 10.1097/SAP.0b013e31815bfeac; Gil, E. S., et al., Functionalized Silk Biomaterials for Wound Healing. Advanced Healthcare Materials, 2013. 2(1): p. 206-217 DOI: 10.1002/adhm.201200192]. Although there are a variety of available hydrogel thickness, the thickness selected in this study was between 100 and 150 μm owing to the limitation of channel size.

### CFD Modeling

The fluid dynamic properties of the 2D multichannel NGC were performed by the ANSYS Fluent (Version 2020 R2) under mixture model theory, which has been typically used to simulate particle-laden flows with low loading [Zhang, L., et al., Investigation on particle motions and resultant impact erosion on quartz crystals by the micro-particle laden waterjet and airjet. Powder Technology, 2020. 360: p. 452-461 DOI: 10.1016/j.powtec.2019.10.032]. The mixture model is designed for multiphase flow and those phases are considered as interpenetrating continua to simulate the diffusion process. Despite volume of fraction (VOF) model being known to be the most commonly used approach of the multiphase simulation, the design objective for immiscible fluids limits its application in this study. Multiphases flow under the mixture model can be solved by calculating a series momentum, continuity and energy equations, as listed below.

The continuity equation for the mixture model is:

$$\frac{\partial }{\partial t} ( {ρ}_{m} ) + ∇ \cdot ( {ρ}_{m} {\vec{v}}_{m} ) = 0$$

where \vec{v}ₘ is the mass-averaged velocity:

$${\vec{v}}_{m} = \frac{{\sum }_{k = 1}^{n} {α}_{k} {ρ}_{k} {\vec{v}}_{k}}{{ρ}_{m}}$$

and ρₘ is the mixture density:

$${ρ}_{m} = {\sum }_{k = 1}^{n} {α}_{k} {ρ}_{k}$$

αₖ is the volume fraction of phase k.

The momentum equation for the mixture model is:

$$\frac{\partial }{\partial t} ( {ρ}_{m} {\vec{v}}_{m} ) + ∇ \cdot ( {ρ}_{m} {\vec{v}}_{m} {\vec{v}}_{m} ) = - {∇}_{p} + ∇ \cdot [ {μ}_{m} ( ∇ {\vec{v}}_{m} + ∇ {\vec{v}}_{m}^{T} ) ] + {ρ}_{m} \vec{g} + \vec{F} + ∇ \cdot ( {\sum }_{k = 1}^{n} {α}_{k} {ρ}_{k} {\vec{v}}_{dr , k} {\vec{v}}_{dr , k} )$$

where n is the number of phases, \vec{F} is body force, and μₘ is the viscosity of the mixture:

$${μ}_{m} = {\sum }_{k = 1}^{n} {α}_{k} {μ}_{k}$$

\vec{v}_{dr,k} is the drift velocity for secondary phase k:

$${\vec{v}}_{dr , k} = {\vec{v}}_{k} - {\vec{v}}_{m}$$

The energy equation for the mixture model is:

$$\frac{\partial }{\partial t} {\sum }_{k = 1}^{n} ( {α}_{k} {ρ}_{k} {E}_{k} ) + ∇ \cdot {\sum }_{k = 1}^{n} ( {α}_{k} {\vec{v}}_{k} ( {ρ}_{k} {E}_{k} + p ) ) = ∇ \cdot ( {k}_{eff} ∇ T ) + {S}_{E}$$

where k_{eff} is the effective conductivity (Σαₖ(kₖ+kₜ)), kₜ is the turbulent thermal conductivity. The first term on the right-hand side of equation represents energy transfer due to the conduction. S_{E} includes any other volumetric heat sources.

$${E}_{k} = {h}_{k} - \frac{p}{{ρ}_{k}} + \frac{{v}_{k}^{2}}{2}$$

for a compressible phase, and Eₖ=hₖ for an incompressible phase, where hₖ is the sensible enthalpy for phase k.

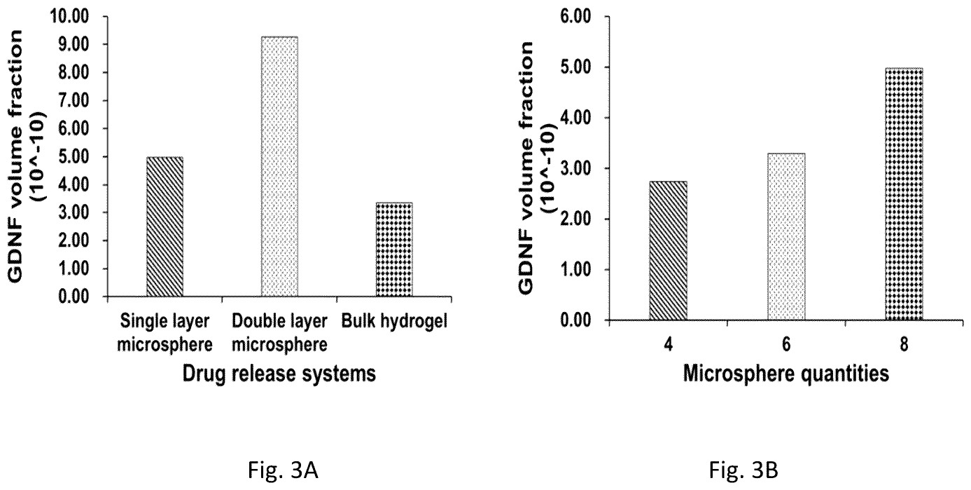

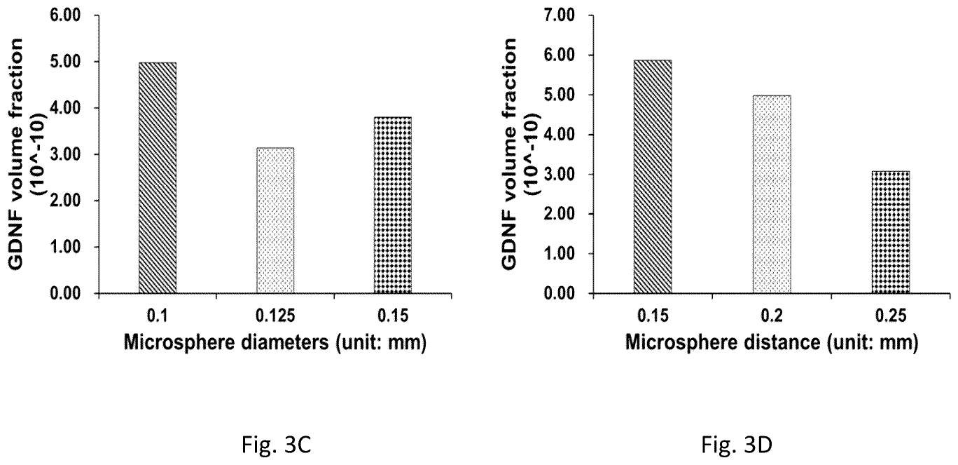

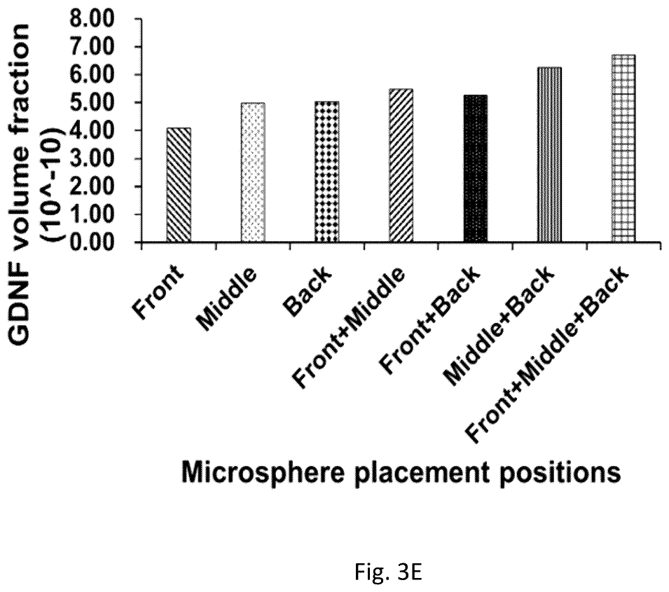

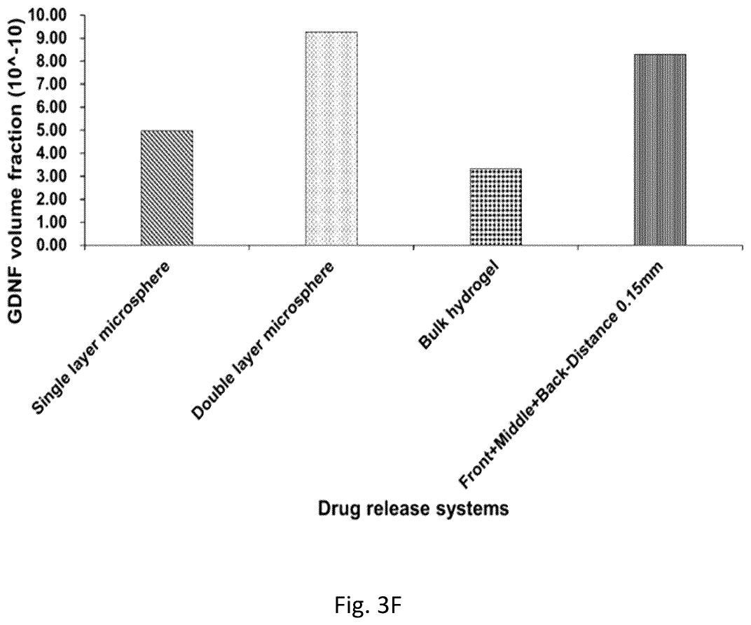

In this work, interstitial fluid and glial cell derived neurotrophic factor (GDNF) were used as two separate phases. Interstitial fluid was chosen to represent the hydrodynamic system around the multichannel NGC and GDNF has been validated as an effective chemical stimulus to enhance the nerve regeneration performance [Lackington, W. A., et al., Controlling the dose-dependent, synergistic and temporal effects of NGF and GDNF by encapsulation in PLGA microparticles for use in nerve guidance conduits for the repair of large peripheral nerve defects. Journal of Controlled Release, 2019. 304: p. 51-64 DOI: 10.1016/j.jconrel.2019.05.001]. Both the phases are treated as Newtonian incompressible fluids with viscosities of 0.0035 and 0.0015 kg/ms, and densities of 1000 and 1370 kg/m³ respectively [Yao, W., Z. Shen, and G. Ding, Simulation of interstitial fluid flow in ligaments: Comparison among Stokes, Brinkman and Darcy models. International Journal of Biological Sciences, 2013. 9(10): p. 1050-1056 DOI: 10.7150/ijbs.7242; Arora, J., et al., Hydrogen exchange mass spectrometry reveals protein interfaces and distant dynamic coupling effects during the reversible self-association of an IgG1 monoclonal antibody. mAbs, 2015. 7(3): p. 525-539 DOI: 10.1080/19420862.2015.1029217; Yao, W., Y. Li, and G. Ding, Interstitial fluid flow: The mechanical environment of cells and foundation of meridians. Evidence-based Complementary and Alternative Medicine, 2012. 2012 DOI: 10.1155/2012/853516; Fischer, H., I. Polikarpov, and A. F. Craievich, Average protein density is a molecular-weight-dependent function. Protein Science, 2004. 13(10): p. 2825-2828 DOI: 10.1110/ps.04688204]. Since there is no direct data on the properties of GDNF, thus viscosity and density of GDNF were estimated from the concentration and molecular weight of GDNF, respectively [Arora, J., et al., Hydrogen exchange mass spectrometry reveals protein interfaces and distant dynamic coupling effects during the reversible self-association of an IgG1 monoclonal antibody. mAbs, 2015. 7(3): p. 525-539 DOI: 10.1080/19420862.2015.1029217; Fischer, H., I. Polikarpov, and A. F. Craievich, Average protein density is a molecular-weight-dependent function. Protein Science, 2004. 13(10): p. 2825-2828 DOI: 10.1110/ps.04688204]. Apart from material properties, four open boundaries were prescribed in the model, including two inlet edges, one outlet edge and wall boundary at the rest of edges (FIG. 3). Inlet 1 was determined as a fixed velocity equal to the physiological interstitial fluid velocity to better mimic the hydrodynamic system around the multichannel NGC, while the velocity of inlet 2 was composed of a burst release (fast speed) and continuous release (slow speed) based on an earlier validated GDNF release profile [Kokai, L. E., A. M. Ghaznavi, and K. G. Marra, Incorporation of double-walled microspheres into polymer nerve guides for the sustained delivery of glial cell line-derived neurotrophic factor. Biomaterials, 2010. 31(8): p. 2313-2322 DOI: 10.1016/j.biomaterials.2009.11.075; Yao, W., Y. Li, and G. Ding, Interstitial fluid flow: The mechanical environment of cells and foundation of meridians. Evidence-based Complementary and Alternative Medicine, 2012. 2012 DOI: 10.1155/2012/853516]. Drug release system with reduced release time and cumulative mass was adopted to examine the growth factor release performance while avoiding the excessive computation cost. Given that the release velocity remains the same when scaling down the release time and cumulative mass at the same proportion, the reduced growth factor release profile would still well represent the release performance for the simulation purpose. Table 1 illustrates how to narrow down the original growth factor release profile.

TABLE 1
Illustration of how to narrow down the growth factor release profile.

| | Total | Burst | Burst | Burst | Continuous | Continuous | Continuous |
|---|---|---|---|---|---|---|---|
| | release | release | release | release | release | release | release |
| | time | mass | time | velocity | mass | time | velocity |
| | (s) | (ng) | (s) | (ng/s) | (ng) | (s) | (ng/s) |
| Original | 5184000 | 5 | 86400 | 5.79*10⁻⁵ | 1.5 | 5097600 | 2.94*10⁻⁷ |
| release | | | | | | | |
| profile | | | | | | | |
| Modified | 519 | 0.0005 | 9 | 5.56*10⁻⁵ | 0.00015 | 510 | 2.94*10⁻⁷ |
| release | | | | | | | |
| profile | | | | | | | |

### GDNF Volume Fraction

The magnitude of GDNF volume fraction was directly given by ANSYS Fluent (Version 2020 R2) to evaluate and compare the GDNF release performance among all the drug release systems. To provide a more comprehensive comparison, two control variables were prescribed, which are constant growth factor mass and constant simulation time. Given a constant GDNF density and release rate, growth factor mass is determined by the surface area of the drug release system and simulation time (release time), making the simulation time a dependent parameter of the surface area. Table 2 lists the surface area of each drug release system as well as the corresponding simulation time. The default simulation time is 519 s, as shown in Table 1, with a time step size of 0.5 s. Due to the long and thin structure of the hydrogel film, its surface area is 166 times that of the default single layer microsphere system. Shortening the simulation time by the same proportion will make the simulation time far less than the unit time step size, therefore, the hydrogel film is not suitable for the comparison of other drug release systems. Thus, the growth factor release performance of hydrogel film with different hydrogel film thicknesses would be compared instead of comparing them with the drug release systems.

TABLE 2
Simulation time of the various drug release systems (MS = Microsphere)

| | | | | | Burst | Continuous | Total |
|---|---|---|---|---|---|---|---|
| Drug | MS | | | Surface | release | release | sim |
| release | diameter | MS | MS | area | time | time | time |
| systems | (mm) | quantity | placement | (mm\hat{ }2) | (s) | (s) | (s) |
| Single layer | 0.1 | 8 | Front/middle/ | 0.251 | 9 | 510 | 519 |
| microsphere | | | back | | | | |
| Single layer | 0.1 | 16 | Front + middle/ | 0.502 | 4.5 | 255 | 259.5 |
| microsphere | | | front + back/ | | | | |
| | | | middle + back | | | | |
| Single layer | 0.1 | 24 | Front + middle + | 0.753 | 3 | 170 | 173 |
| microsphere | | | back | | | | |
| Single layer | 0.125 | 8 | Middle | 0.393 | 6 | 326.5 | 332.5 |
| microsphere | | | | | | | |
| Single layer | 0.15 | 8 | Middle | 0.565 | 4 | 226.5 | 230.5 |
| microsphere | | | | | | | |
| Single layer | 0.1 | 6 | Middle | 0.188 | 12 | 680 | 692 |
| microsphere | | | | | | | |
| Single layer | 0.1 | 4 | Middle | 0.126 | 18 | 1020 | 1038 |
| microsphere | | | | | | | |
| Double layer | 0.1 | 16 | Middle | 0.502 | 4.5 | 255 | 259.5 |
| microsphere | | | | | | | |
| Bulk | 0.5 | 1 | Middle | 0.785 | 3 | 163 | 166 |
| hydrogel | | | | | | | |

The results are now described.

### A ‘Relay’ Type NGC Design

This study is the first to introduce a ‘relay’ type NGC design, which provides three cavities to load the drug release system, connected by two multichannel NGCs. One of the biggest factors affecting the NGC nerve regeneration performance is insufficient nerve cell concentrations and sustained nerve growth factor availability NGCs targeting long nerve gap injuries (>4 cm). The intermediate cavities provided could be integrated with drug release systems and culture of nerve related cells [Fadia, N. B., et al., Long-gap peripheral nerve repair through sustained release of a neurotrophic factor in nonhuman primates. Science Translational Medicine, 2020. 12(527) DOI: 10.1126/scitranslmed.aav7753; Rodríguez, F. J., et al., Nerve guides seeded with autologous Schwann cells improve nerve regeneration. Experimental Neurology, 2000. 161(2): p. 571-584 DOI: 10.1006/exnr. 1999.7315]. Unlike the commonly used multichannel NGC, the extra intermediate cavity of the ‘relay’ type NGC can be used as a relay to further enhance the cell concentration as well as the nerve regeneration performance by loading drug release systems or appropriate cells. Thus, the application of ‘relay’ type NGC has the potential to improve the effective regeneration length of NGC to cater for long nerve gap injuries, which bears a huge clinical significance.

### GDNF Volume Fraction Under the Assumption of Constant Simulation Time

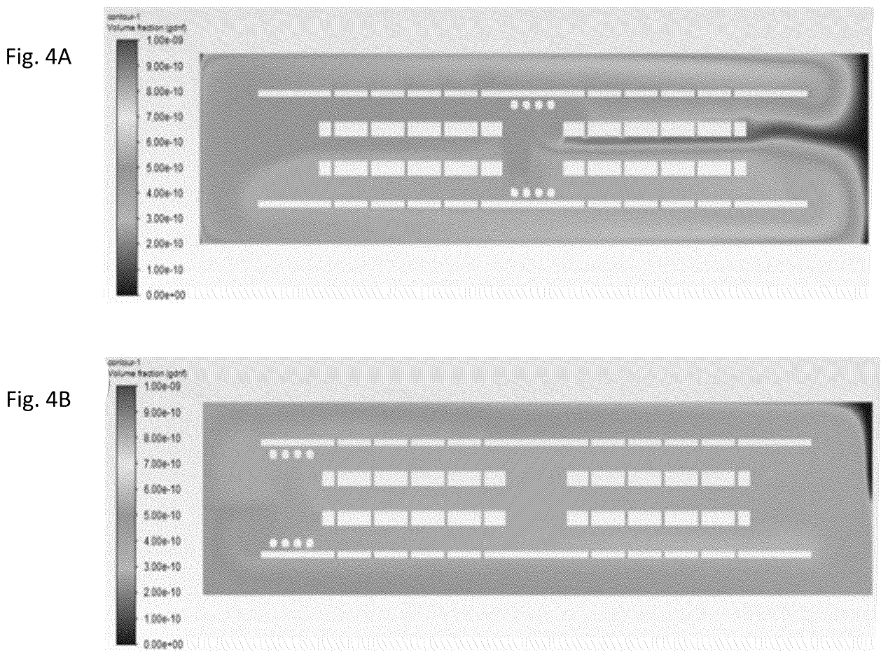

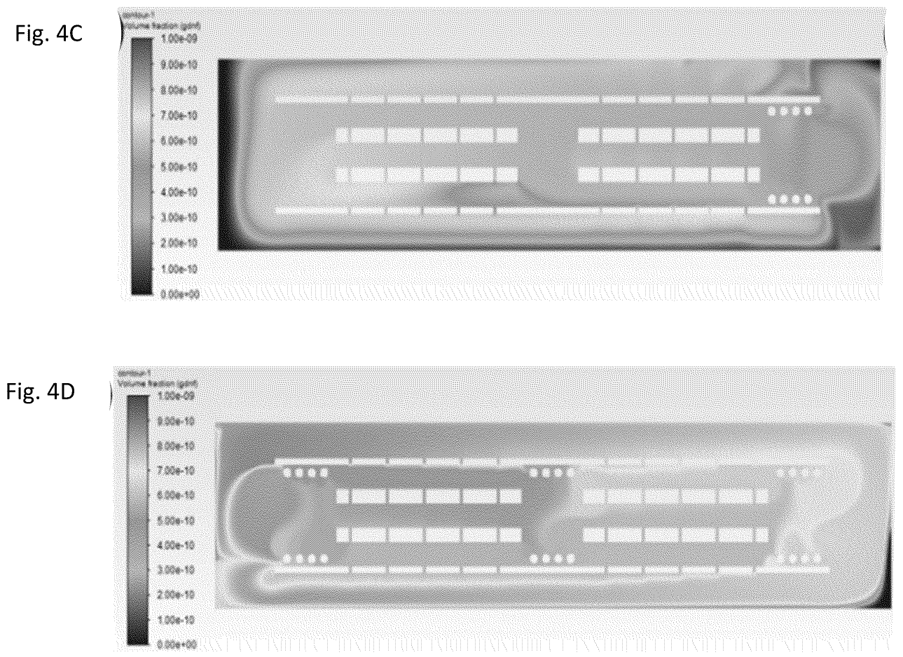

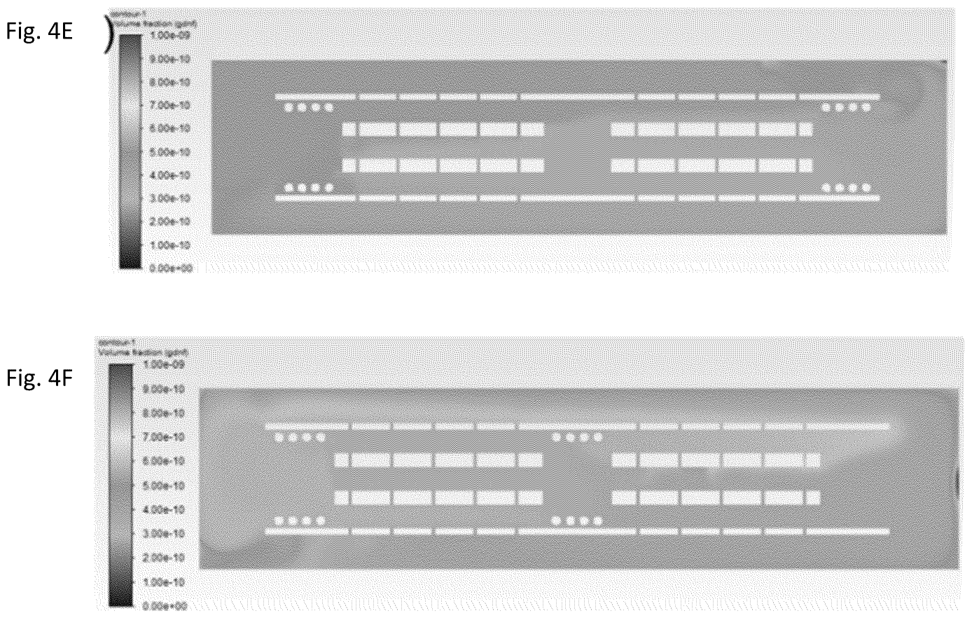

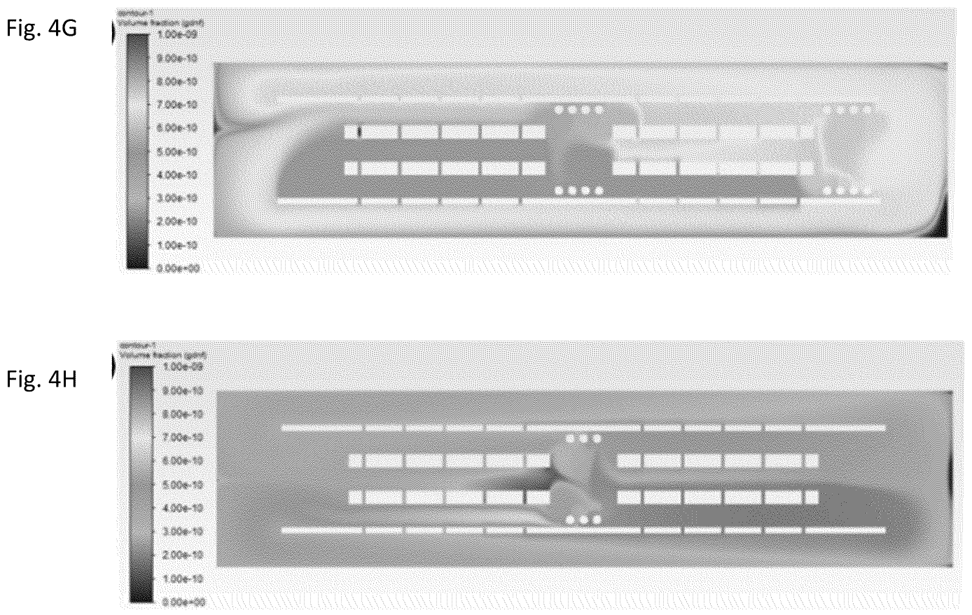

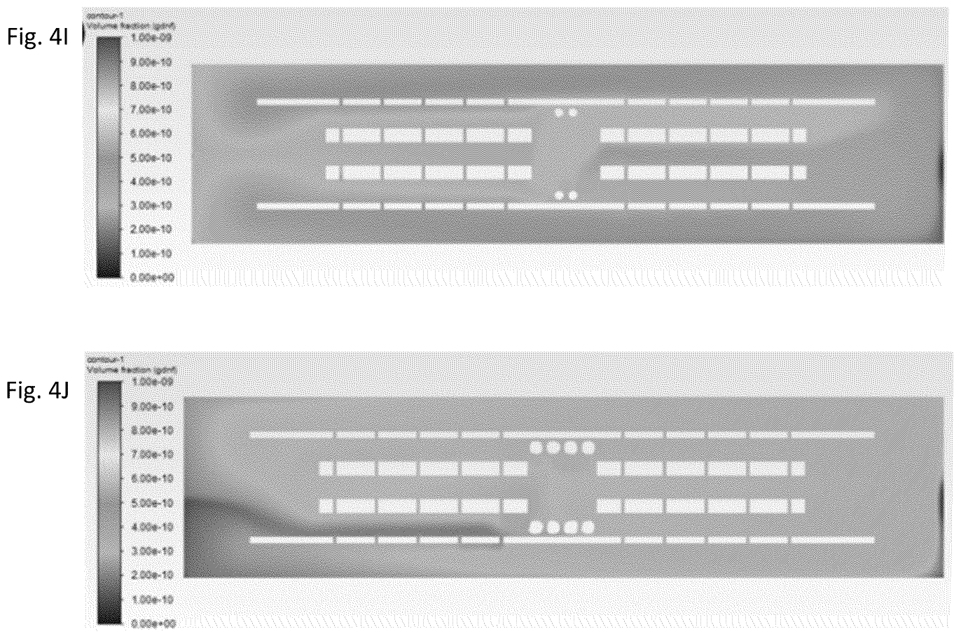

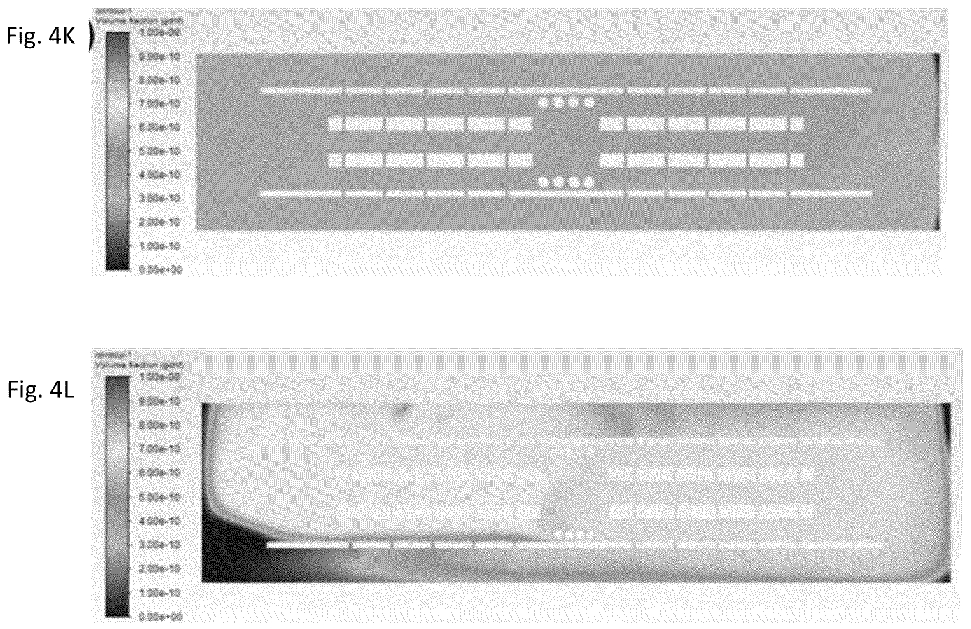

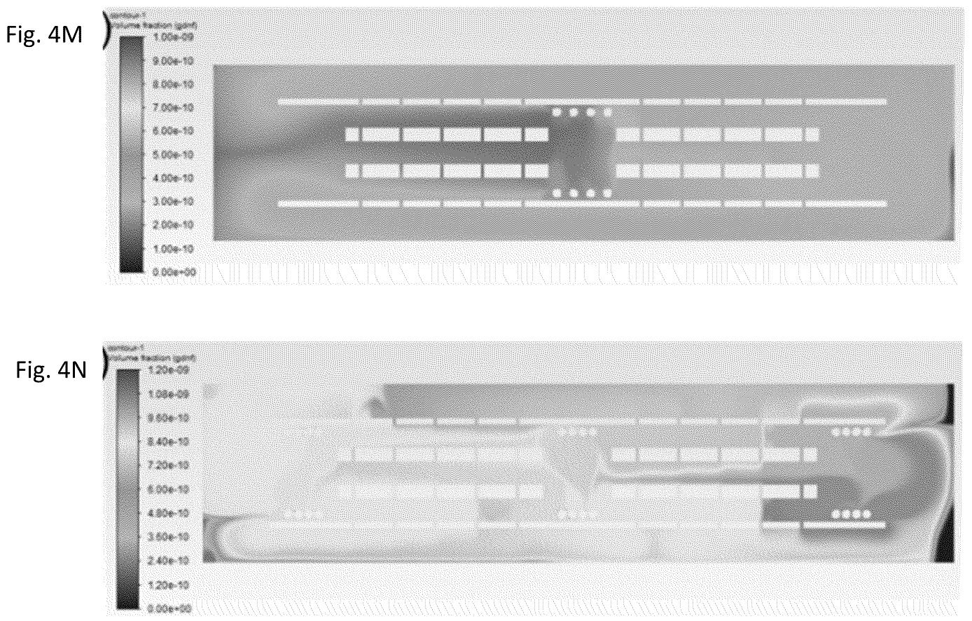

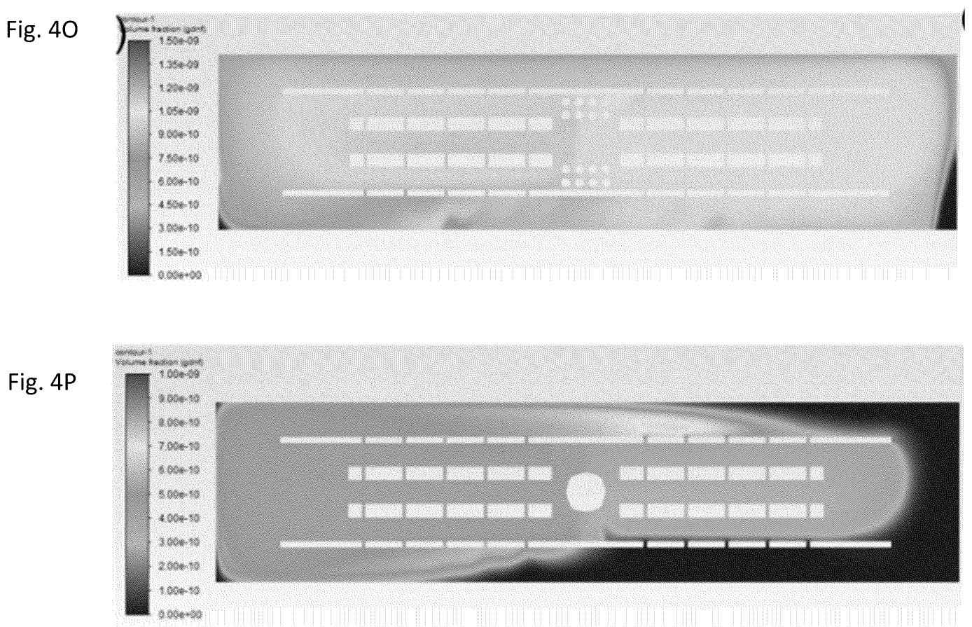

FIG. 3A and FIG. 3F shows the influence of different drug release systems and FIG. 3B-FIG. 3E shows microspheres features on GDNF volume fractions. The simulation time was 519 s with 9 s burst release and 510 s continuous release and identical simulation time was applied to all the models to evaluate the GDNF volume fraction at the last time step. From FIG. 3A, it can be found that double layer microsphere (9.27*10⁻¹⁰) achieved the highest GDNF volume fraction and the magnitude is roughly 1.8 and 3 times compared with single layer microsphere (4.98*10⁻¹⁰) and bulk hydrogel systems (3.34*10⁻¹⁰) respectively. FIG. 3B to 3E reflected the effect of the microsphere features on GDNF volume fraction in a single layer microsphere system. It can be seen that 8 microspheres, 0.1 mm diameter, 0.15 mm adjacent distance and front-middle-back placement can achieve higher GDNF volume fraction from each group. Therefore, by combining those suggested settings, it is supposed to raise the GDNF release performance to a next level. The combined model was evaluated again and the corresponding result was shown in the last column of FIG. 3F. According to FIG. 3F, it can be seen that double layer microsphere system still holds the best performance among all the drug release systems. However, the increase between the default single layer microsphere system and combined model is over 60% by changing microsphere features from multiple dimensions. FIG. 4A to FIG. 4P shows the GDNF distribution among all the models at the last time step.

### GDNF Volume Fraction Under the Assumption of Constant Growth Factor Mass

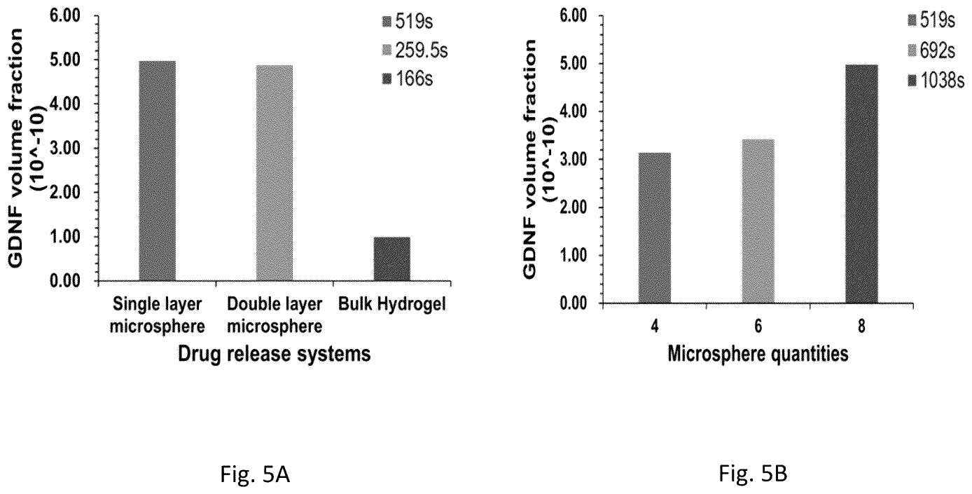

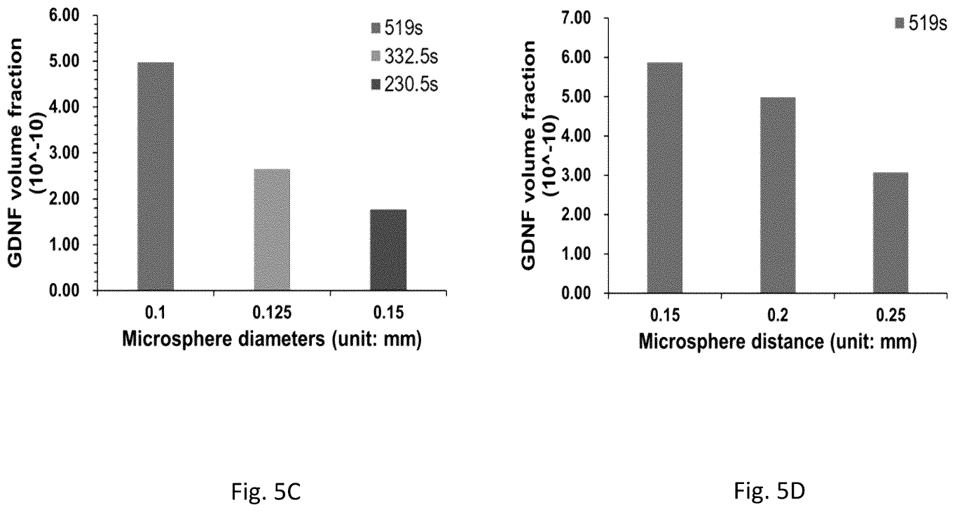

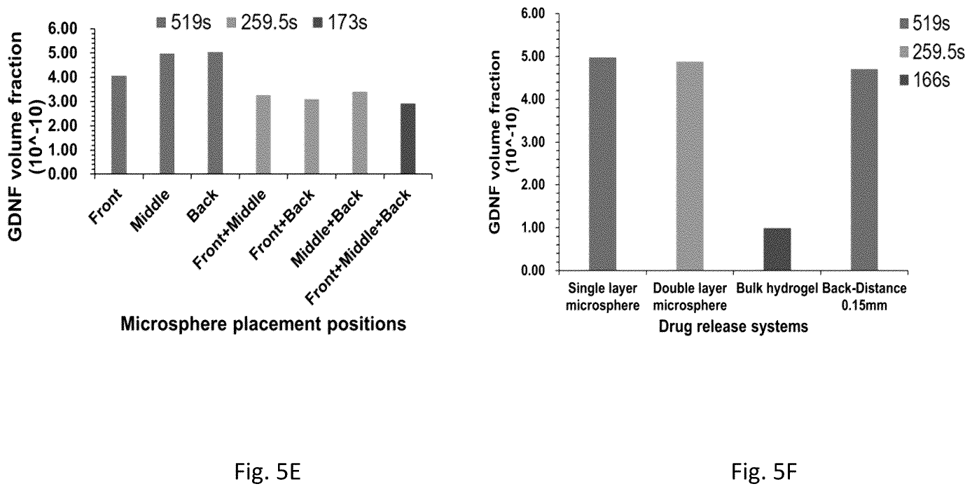

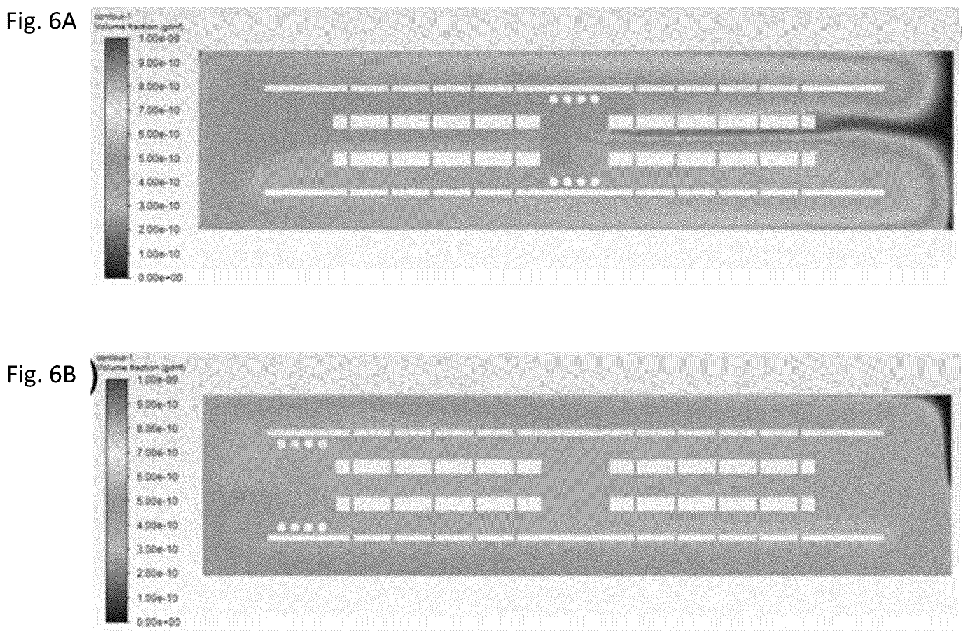

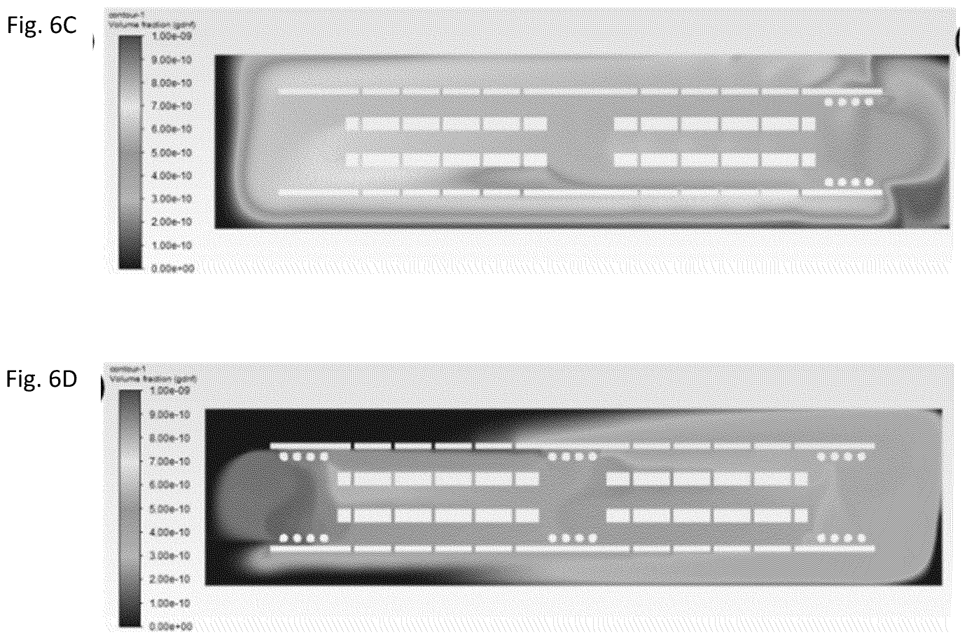

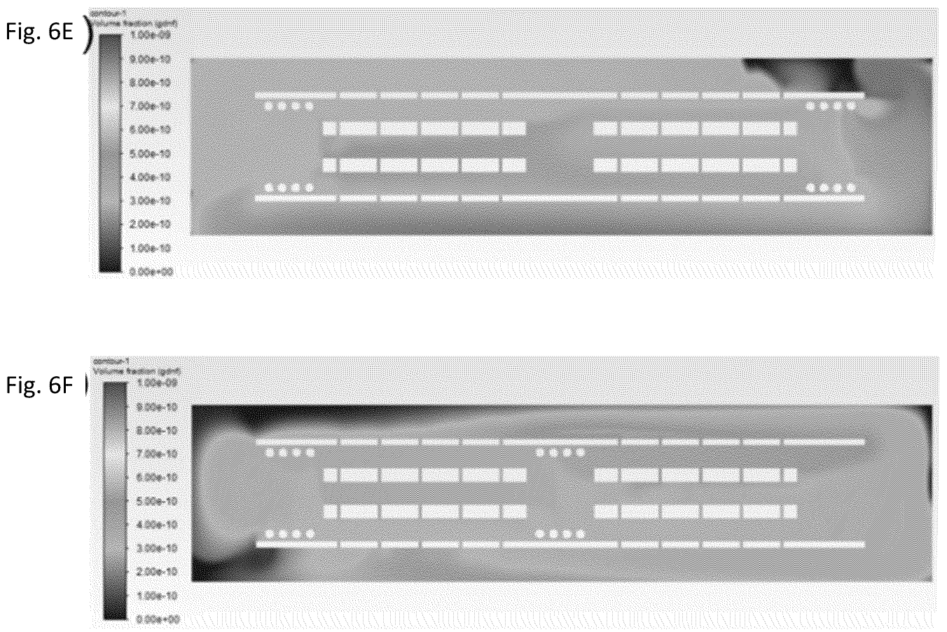

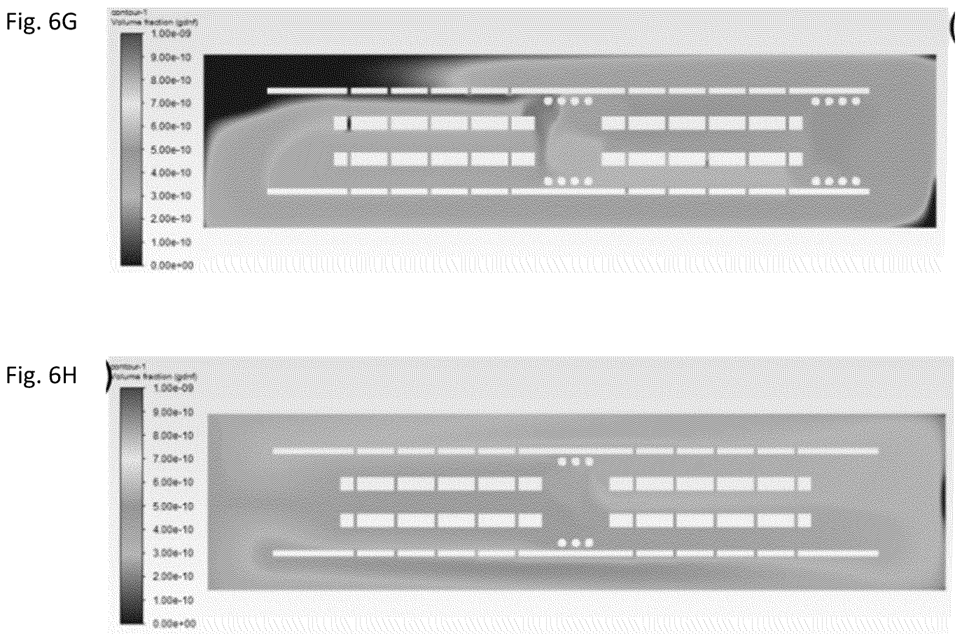

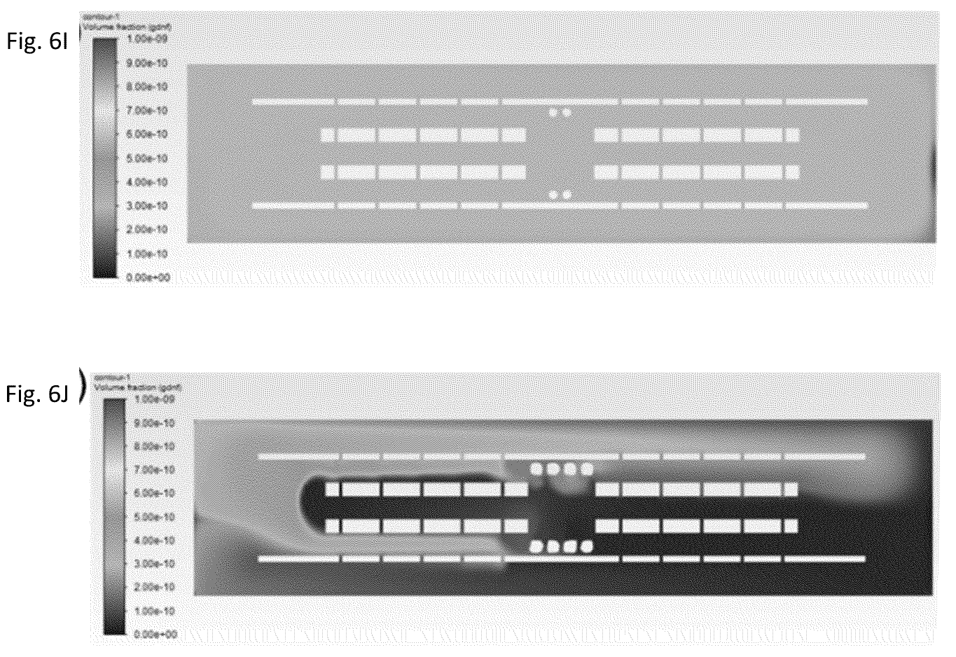

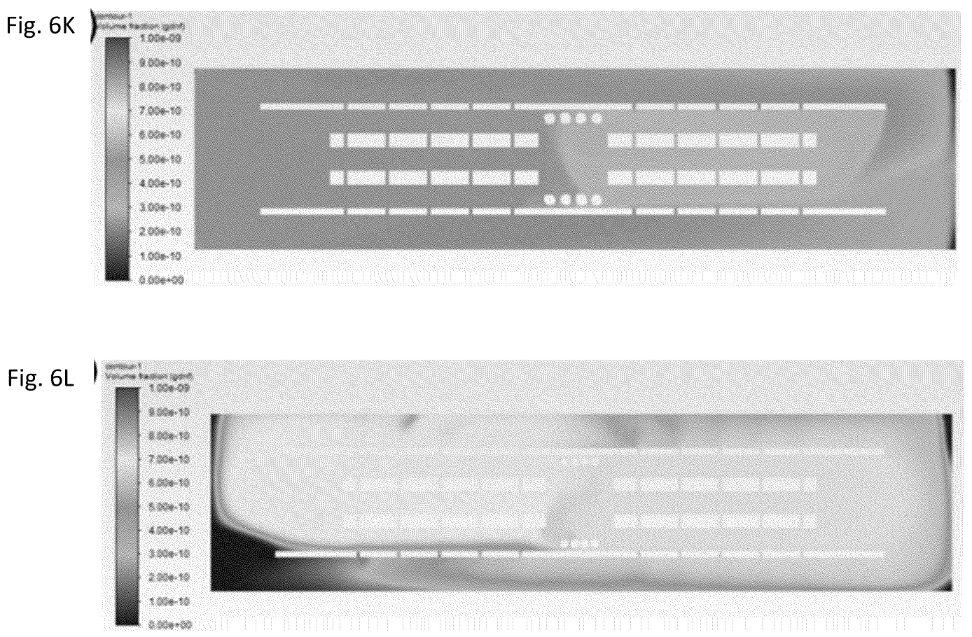

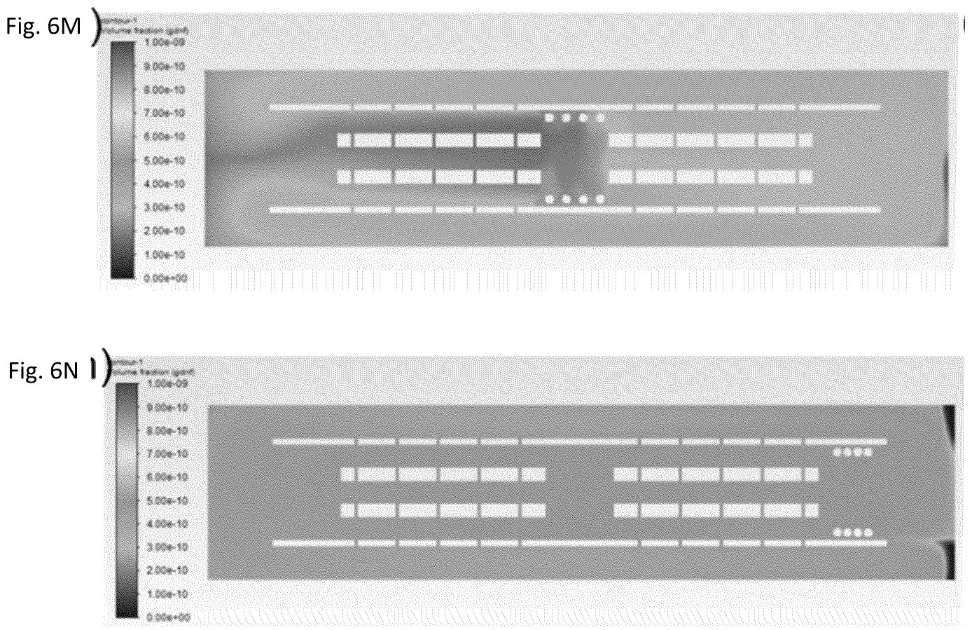

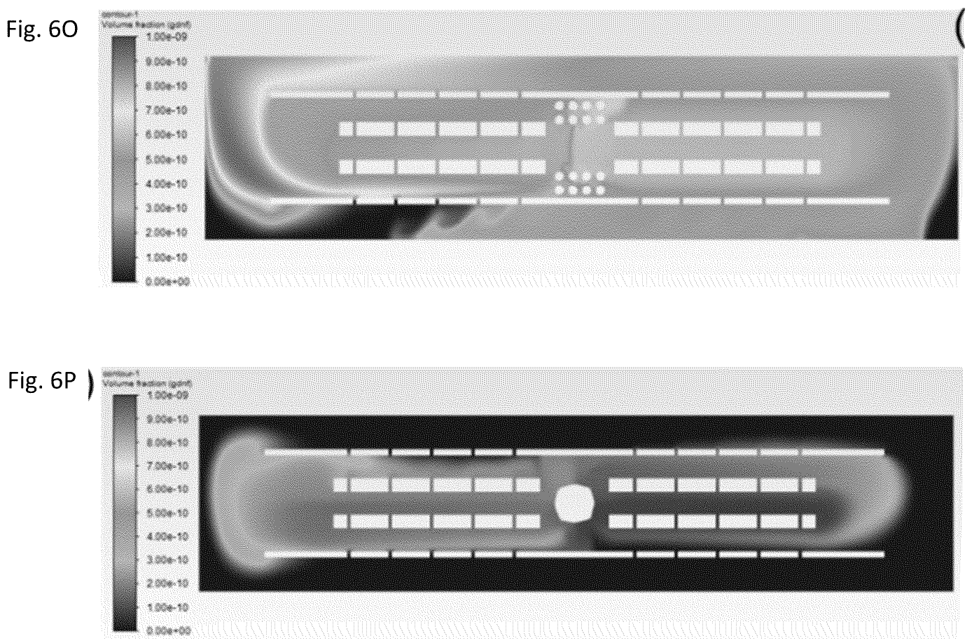

To keep constant growth factor, simulation time was adjusted based on the surface area of drug release system, which is illustrated in Table 2. GDNF volume fraction and distribution among different drug release systems are shown in FIG. 5A to FIG. 5F and FIG. 6A through FIG. 6P, respectively. From FIG. 5A, it can be seen that single layer microsphere system (4.98*10⁻¹⁰) and double layer microsphere system (4.88*10⁻¹⁰) have a comparable GDNF volume fraction, which is much higher than that of bulk hydrogel system (9.90*10⁻¹¹). FIG. 5B to FIG. 5E illustrates the effect of different microsphere features (quantity, diameter, adjacent distance, placement locations) on GDNF volume fraction in single layer microsphere system. Considering the surface area remains constant when the distance between microspheres is changed, the simulation time of the models in FIG. 5D equal to 519 s, which is the default simulation time. From FIG. 5B to FIG. 5E, it can be found that 8 microspheres, 0.1 mm diameter, 0.15 mm distance and back placement achieve the highest GDNF volume fraction under each experiment group, respectively. Therefore, FIG. 5F is constructed by integrating those preferred settings into one drug release system, then calculating the GDNF release performance and comparing it with three predetermined drug release systems. From FIG. 5F, it can be seen that the GDNF volume fraction can be slightly increased by the microsphere movement from middle to back and the denser arrangement of microspheres (0.15 mm adjacent distance). However, the combined model (4.71*10⁻¹⁰) which integrated both back placement and 0.15 mm microsphere distance even results in a worse performance compared with solely adjusting the microsphere position to back and distance to 0.15 mm in FIG. 5E and FIG. 5D, respectively.

### GDNF Volume Fraction Among Different Hydrogel Films

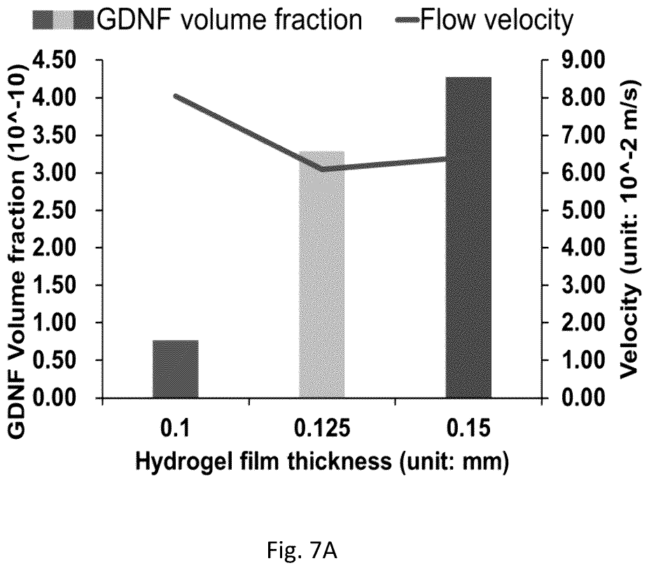

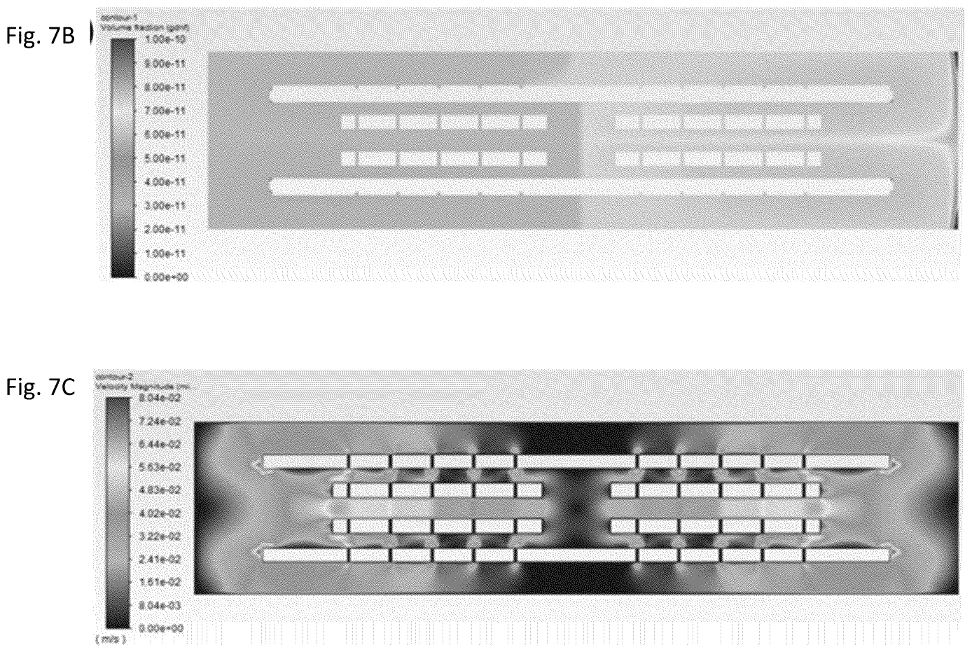

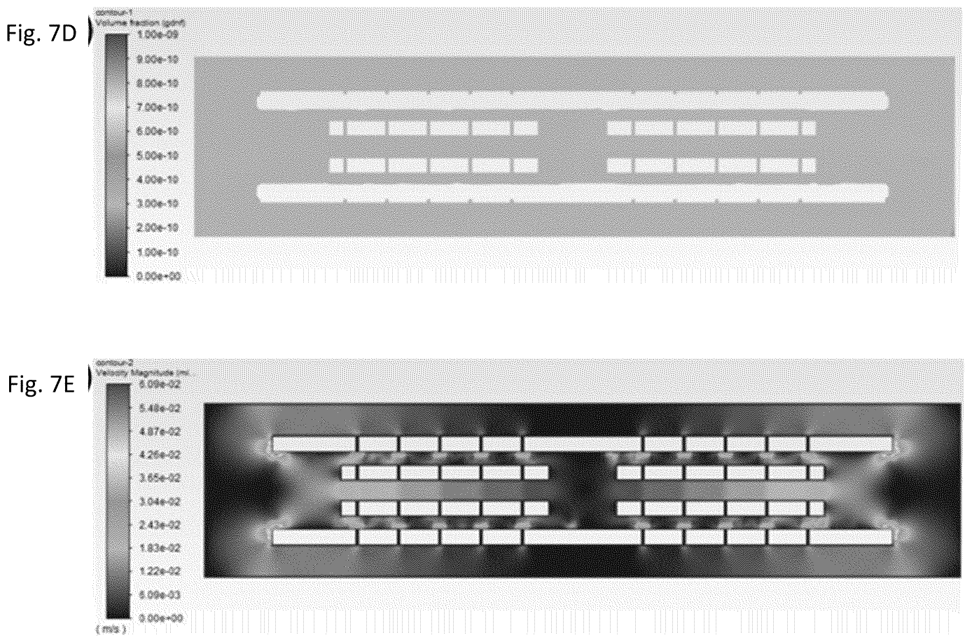

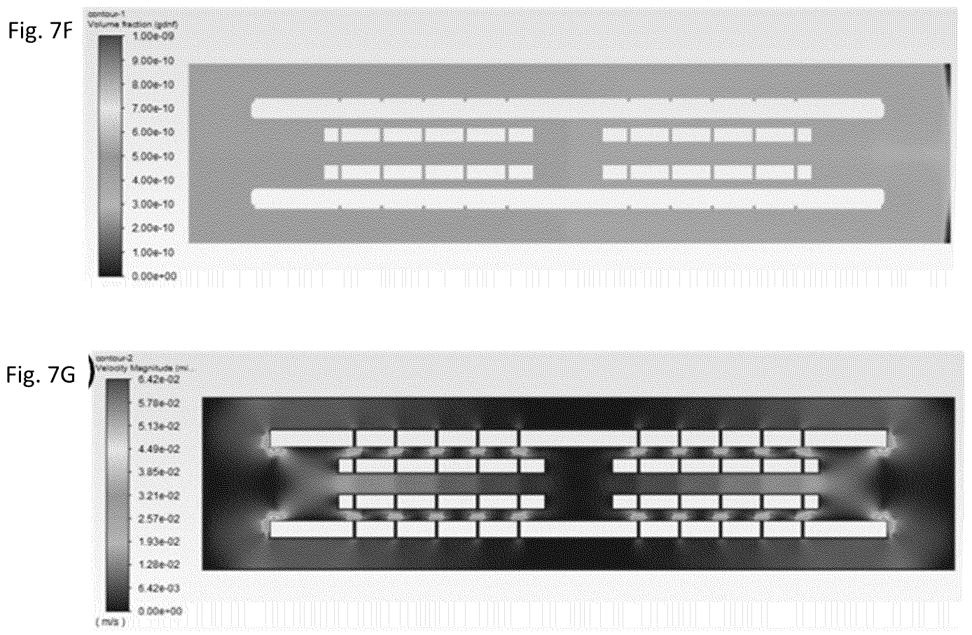

The influence of different hydrogel film thicknesses on GDNF volume fraction was examined and shown in FIG. 7A. A uniform simulation time of 519 s (9 s burst release and 510 s continuous release) was applied to all hydrogel films model. From FIG. 7A, it can be seen that when the film thickness increased from 0.1 mm (7.69*10⁻¹¹) to 0.125 mm (3.29*10⁻¹⁰), the GDNF volume fraction increased by 328%, while when the film thickness increased from 0.125 mm to 0.15 mm (4.28*10⁻¹⁰), the GDNF volume fraction increased only by 30%. Furthermore, the relationship between flow velocity and GDNF volume fraction is not monotonically increasing, which illustrates that higher velocity can not only facilitate the diffusion of growth factors, but also accelerate the mixture fluid (including growth factor) flowing out of the structure. FIG. 7B to 7G shows the GDNF distribution and velocity magnitude of all the hydrogel film models. Similar GDNF distributions are found in all of the models with a gradually increasing GDNF volume fraction from 7.69*10⁻¹¹ to 4.28*10⁻¹⁰. The highest velocity is achieved around the inner edges at both proximal and distal sides of the multichannel NGC for all the hydrogel films.

In most of the growth factor embedded scaffolds, microspheres and hydrogels are the two most commonly used drug carriers owing to the well-developed fabrication method and controllable release profile. However, the variation of microsphere and hydrogel features is usually limited, leading to a lack of evaluation on the performance of different microsphere and hydrogel-based drug release systems. Therefore, in this study, the effect of different drug release systems on growth factor (GDNF) volume fraction in a multichannel NGC was carried out by controlling a constant simulation time and growth factor mass. Furthermore, growth factor embedded hydrogel films with different thicknesses were simulated to find out the most efficient model.

Note that for both microsphere and hydrogel, the material should have proper biodegradability because the growth factor is sealed inside and supposed to be released along with the degradation process, thus the release time of growth factors is usually determined by the degradability of the carrier materials. Furthermore, the release time is limited to less than 60 days in most of the cases, thereby it is important to release and maintain a high growth factor concentration under a fixed release time. The total simulation time was scaled down from an existed GDNF release profile at the same proportion, which was 519 s composed of 9 s burst release and 510 s continuous release [Kokai, L. E., A. M. Ghaznavi, and K. G. Marra, Incorporation of double-walled microspheres into polymer nerve guides for the sustained delivery of glial cell line-derived neurotrophic factor. Biomaterials, 2010. 31(8): p. 2313-2322 DOI: 10.1016/j.biomaterials.2009.11.075]. FIG. 3A to 3F and FIG. 4A to 4P shows the magnitude and distribution of GDNF among different drug release systems at the last time step. The figures illustrate that double layer microsphere system achieves the highest GDNF volume fraction compared to the other two drug release systems. The huge increase can be attributed to twice as many microspheres, which provide more tunnels to deliver the growth factor into the scaffolds. In the single layer microsphere system, the GDNF volume fraction was significantly changed by adjusting the microsphere features, including microsphere diameter, quantity, adjacent distance and locations. The combination of smaller adjacent distance (0.15 mm) and multiple placements (front+middle+back) of microspheres improved the GDNF volume fraction by 67% from 4.98*10⁻¹⁰ to 8.3*10⁻¹⁰. Although more microspheres were implanted in the combined group than double layer microsphere system, which was supposed to achieve a higher GDNF volume fraction, the double layer microsphere system still held the best performance under a constant simulation time. The higher GDNF volume fraction of double layer microsphere system probably benefits from the small distance between two layers, which could act as a flow accelerator to speed up the flow and deliver the growth factor more efficiently to the whole structure. Therefore, the result shows that quantity and adjacent distance (both vertically and horizontally) of microspheres have the maximum impact on the GDNF volume fraction, but other factors including microsphere diameter and locations could also influence the GDNF concentration.

Since the NGC needs to connect with the original nerve in human body, the diameter of NGC and human nerve must be the same. Thus, it can be inferred that tiny lumen size is achieved, therefore leading to a limited number of embedded growth factors in the NGC. The growth factor mass is controlled by the drug release system surface area and simulation (release) time. Higher surface area would result in shorter simulation time and vice versa. FIG. 5A to FIG. 5F and FIG. 6A to 6p shows the GDNF magnitude and distribution in a variety of drug release systems. The double layer microsphere system does not achieve a superior performance as it is under the assumption of constant simulation time, but has a GDNF volume fraction comparable to that of the single layer microsphere system. For the single layer microsphere system, decreasing the microsphere quantity and increasing the adjacent distance would cause a disadvantageous GDNF volume fraction, which is in agreement with the NGC groups under the assumption of constant simulation time. From FIG. 5E, it can be seen that back position slightly improved the GDNF volume fraction compared to the middle position. However, the GDNF volume fraction of the combined model is lower than that before the combination, as it is shown in FIG. 5F. Considering the even distribution of GDNF in FIG. 6N, it is possible that the structure reaches the steady state between the GDNF and interstitial fluid before the end of the simulation, thereby allowing the excessive growth factor to flow out of the structure and decreasing the GDNF volume fraction. The reduced GDNF volume fraction of the combined model reveals that delaying the time to steady state of the drug release system is crucial to maintain high growth factor concentrations.

The effect of different hydrogel film thicknesses on the GDNF volume fraction was evaluated in this study and the GDNF magnitude as well as distribution are shown in FIG. 7A to FIG. 7G. Note that for hydrogel film systems, the surface area is more than 150 times higher than the other three drug release systems, therefore it is meaningless to compare the GDNF volume fraction with other drug release systems. As can be seen from FIG. 7A to FIG. 7G, thicker hydrogel film can contribute to higher GDNF volume fraction, and similar GDNF and velocity distributions are found from all the hydrogel films. The poor volume fraction of GDNF in the 0.1 mm hydrogel film can be attributed to the high flow velocity, which accelerated the flow of GDNF out of the structure, but failed to efficiently transport and maintain GDNF in the whole structure, especially in the hydrogel film system. As the same parameter can lead to opposite effects in different drug release systems, it is particularly important to evaluate the growth factor release performance among various drug release systems.

Under the assumptions of constant simulation time and growth factor mass, double layer microsphere system possesses excellent GDNF release performance, which can be used as an ideal drug release system for multichannel NGC. However, the thickness of double layer microsphere system (>200 μm) could take up more than half of the channel size (340 μm), resulting in the potential risk of blocking the peripheral channels of the multichannel NGC. Thus, it is necessary to balance the thickness of the drug release system and channel diameter. Due to the easy implantation of hydrogel film and even distribution of GDNF throughout the whole structure, (which is crucial to guide the nerve regeneration at the same rate) hydrogel film system can be regarded as a strong candidate in drug release systems.

This study utilized CFD simulation approach to examine the growth factor diffusion process in drug release system embedded multichannel NGCs. Two commonly used carriers and four different drug release systems including single layer microsphere system, double layer microsphere system, bulk hydrogel and hydrogel film were chosen, and the effect of microsphere features and hydrogel thicknesses on the GDNF volume fraction and distribution were evaluated. Under the assumptions of constant simulation time and constant growth factor mass, double layer microsphere system achieves excellent GDNF release performance, therefore it can be regarded as the best choice among all the drug release systems. The ratio of channel diameter to the thickness of the double layer microsphere system can be balanced before integrating the drug release system to prevent blockage of the peripheral channels. For single layer microsphere system, the combination of smaller adjacent distance (0.15 mm) and multiple positions (front+middle+back) of microspheres improved the GDNF volume fraction by 67% under the assumption of constant simulation time. Although the GDNF volume fraction can be increased by moving the microspheres from middle to back and decreasing the adjacent distance from 0.2 mm to 0.15 mm under the assumption of constant growth factor mass, the combined model quickly reached the steady state owing to the rapid flow velocity, which leads to the decrease of GDNF volume fraction of the combined model. Therefore, it is important to prolong the time for the drug release system to reach steady state to maintain a high GDNF concentration. Furthermore, GDNF volume fraction increased monotonously with the increase of the thickness of hydrogel film, and hydrogel film with 0.15 mm thickness achieves the best performance without blocking the channels.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A nerve guidance conduit device, comprising:
a tubular outer housing having an inner lumen; and
a plurality of nerve-holding sections positioned within the inner lumen of the housing, each nerve-holding section comprising one or more channels each configured to receive a nerve;
wherein the inner lumen of the housing is divided into an alternating series of nerve-holding sections and inner lumen cavities.

2. The device of claim 1, wherein the outer housing and the plurality of nerve-holding sections comprises a plurality of pores, such that each channel and each inner lumen cavity is fluidly connected to each other and to an exterior of the outer housing.

3. The device of claim 1, further comprising at least one therapeutic-releasing substrate positioned within the inner lumen.

4. The device of claim 3, wherein the substrate is chosen from a group consisting of: microparticles, bulk hydrogel and hydrogel film.

5. The device of claim 3, wherein the substrate is loaded with a therapeutic selected from the group consisting of: growth factors, neurotrophic factors, cell adhesion molecules, proteins, peptides, small molecules, nucleic acid molecules, cytokines, stem cells, Schwann cells, and upregulators of regeneration-associated genes.

6. The device of claim 5, wherein the growth factor is glial cell line-derived neurotrophic factor (GDNF).

7. The device of claim 3, wherein the substrate is configured to release the therapeutic over a period of time.

8. The device of claim 3, wherein the substrate is configured to release the therapeutic over a period of at least 50 days.

9. The device of claim 1, wherein the one or more channels have diameters ranging from 50 μm to 1000 μm.

10. The device of claim 1, wherein the outer housing has a diameter of at least 0.1 mm.

11. The device of claim 1, wherein the outer housing has a length of a least 3 mm.

12. The device of claim 4, wherein the hydrogel film has a thickness between 0.1 mm and 0.2 mm.

13. The device of claim 4, wherein the microparticles comprise microspheres with diameters of about 100 μm.

14. The device of claim 4, wherein the bulk hydrogel has a thickness of at least 25 μm.

15. The device of claim 4, wherein the bulk hydrogel has a diameter of at least 100 μm.

16. The device of claim 3, wherein the at least one therapeutic-releasing substrate is positioned in the inner lumen in a location chosen from a front, middle and/or back locations.

17. The device of claim 1, wherein the device comprises a material selected from the group consisting of: poly(lactic co-glycolic acid) (PGA), poly(1-lactic-acid) (PLA), polycaprolactone (PCL), poly(ethylene glycol) diacrylate (PEGDA), gelatin methacryloyl (GelMA), polydimethylsiloxane (PDMS), collagen, chitosan, decellularized extracellular matrix (dECM), cellulose, silk, and ionic liquids.

18. The device of claim 1, further comprising a coating on at least a portion of the device.

19. The device of claim 18, wherein the coating is a material selected from the group consisting of: conductive materials, ferromagnetic materials, thermoelectric materials, nanoparticles, 2D materials such as graphene, MXenes, conductive polymers and hydrogels.

20. A method of treating nerve injury comprising: implanting the device of claim 1 at the site of a nerve injury; thereby treating the injury.
